SEQUENCE LISTING (14 sequences)

SEQ ID NO: 1 - DNA, 1768 bp, Porcine circovirus 2
  misc_feature (1)..(1768): Porcine circovirus 2 isolate PCV2-Ha08, complete genome
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcacctccaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatcgagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgggactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaattgtttttattattcatttagggtttaagtggggggtctttaagattaaattctctgaattgtacatacattgttacacggatattgtagtcctggtcgtatttactgttttcgaacgcagtgccgaggcctacgtggtccacatttctactggtttggagtctcatccacagctgattccttttgttatttggttggaagtaatcaatagtggaatcaagaacaggtttgggggtaaagtagcgggagtggtaggagaagggctgggttatggtatggcgggaggagtagttaacataggggtcataggtgagggctgtggcttttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttttgactgtggttcgcttgatagtatatccgaaggtgcgggagaggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 2 - protein, 314 aa, Porcine circovirus 2
MPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLIECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLGLCDRYPLTVETKGGTVPFLARSILITSNQTPLEWYSSTAVPAVEALYRRITSLVFWKNATEQSTEEGGQFVTLSPPCPEFPYEINY

SEQ ID NO: 3 - protein, 233 aa, Porcine circovirus 2
MTYPRRRYRRRRHRPRSHLGQILRRRPWLVHPRHRYRWRRKNGIFNTRLSRTFGYTIKRTTVKTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWMRLQTSRNVDHVGLGTAFENSKYDQDYNIRVTMYVQFREFNLKDPPLKP

SEQ ID NO: 4 - protein, 104 aa, Porcine circovirus 2
MVTIPPLVSRWFPVCGFRVCKISSPFAFTTPRWPHNDVYISLPITLLHFPAHFQKFSQPAEISDKRYRVLLCNGHQTPALQQGTHSSRQVTPLSLRSRSSTLDQ

SEQ ID NO: 5 - DNA, 22 bp, Artificial
  inverse primer
agctccacactcgatcagtaag

SEQ ID NO: 6 - DNA, 23 bp, Artificial
  inverse primer
cctagatctcagggacaacggag

SEQ ID NO: 7 - DNA, 1768 bp, Porcine circovirus 2
  misc_feature (1)..(1768): Porcine circovirus 2 isolate PCV2-Ha09, complete genome
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcaccttcaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatggagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccatgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaattgtttttattattcatttagggtttaagtggggggtctttaagattaaattctctgaattgtacatacattgttacacggatattgtagtcctggtcgtatttactgttttcgaacgcagtgccgaggcctacgtggtccacatttctactggtttggagtctcatccacagctgattccttttgttatttggttggaagtaatcaatagtggaatcaagaacaggtttgggggtaaagtagcgggagtggtaggagaagggctgggttatggtatggcgggaggagtagttaacataggggtcataggtgagggctgtggcttttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttttgactgtggttcgcttgatagtatatccgaaggtgcgggagaggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 8 - protein, 314 aa, Porcine circovirus 2
MPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKNATEQSTEEGGQFVTLSPPCPEFPYEINY

SEQ ID NO: 9 - protein, 233 aa, Porcine circovirus 2; identical to SEQ ID NO: 3

SEQ ID NO: 10 - protein, 104 aa, Porcine circovirus 2
MVTIPPLVSRWFPVCGFRVCKISSPFAFTTPRWPHNDVYISLPITLLHFPAHFQKFSQPAEISDKRYRVLLCNGHQTPALQQGTHSSRQVTPLSLRSRSSTLHQ

SEQ ID NO: 11 - DNA, 1767 bp, Porcine circovirus 2
  misc_feature (1)..(1767): Porcine circovirus 2 isolate PCV2-Ha10, complete genome
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctatttgattattttattgttggcgaggagggtaatgaggaaggacgaacacctcaccttcaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatcgagtgtggagctcctagatctcagggacaacggagtgacctgtctactgctgtgagtaccttgttggcgagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccatgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctgatttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttttgactgtggttcgcttgatagtatatccgaaggtgcgggagaggcgggtgttgaagatgccatttttccttctccagcggcaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 12 - protein, 314 aa, Porcine circovirus 2
MPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLIECGAPRSQGQRSDLSTAVSTLLASGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKNATEQSTEEGGQFVTLSPPCPEFPYEINY

SEQ ID NO: 13 - protein, 233 aa, Porcine circovirus 2
MTYPRRRYRRRRHRPRSHLGQILRRRPWLVHPRHRCRWRRKNGIFNTRLSRTFGYTIKRTTVKTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 14 - protein, 104 aa, Porcine circovirus 2
MVTIPPLVSRWFPVCGFRVCKISSPFAFTTPRWPHNDVYISLPITLLHFPAHFQKFSQPAEISDKRYRVLLCNGHQTPARQQGTHSSRQVTPLSLRSRSSTLDQ

(19) United States
(12) Patent Application Publication
(10) Pub. No.: US 2013/0251719 A1
(43) Pub. Date: Sep. 26, 2013

(54) BONE MARROW APLASIA WITH HAEMORRHAGIC DISEASE IN CALVES CAUSED BY NOVEL PATHOGEN

(71) Applicants: Hermann Muller, Leipzig (DE); Mohammad Yahya Halami, Leipzig (DE); Jens Bottcher, Dorfen (DE); Eva Kappe, Poing (DE); Benjamin Schade, Lechbruck am See (DE)

(72) Inventors: Hermann Muller, Leipzig (DE); Mohammad Yahya Halami, Leipzig (DE); Jens Bottcher, Dorfen (DE); Eva Kappe, Poing (DE); Benjamin Schade, Lechbruck am See (DE)

(73) Assignees: Tiergesundheitsdienst Bayern E.V., Poing-Grub (DE); Universitat Leipzig, Leipzig (DE)

(21) Appl. No.: 13/503,147

(22) PCT Filed: Oct. 22, 2010
(86) PCT No.: PCT/EP10/65983

§ 371 Date: Jul. 5, 2012

(30) Foreign Application Priority Data
Oct. 22, 2009 (EP) 09173810.4

(51) Int. Cl. C07K 14/01; C07K 7/08; C07K 7/06; C12N 7/00

(52) U.S. Cl. CPC C07K 14/01; C12N 7/00; C07K 7/08; C07K 7/06
USPC 424/139.1; 435/235.1; 536/23.72; 435/325; 424/186.1; 530/350; 530/387.9; 435/5; 530/324; 530/325; 530/326; 530/327; 530/328; 530/329

(57) ABSTRACT

The present invention refers to a novel circovirus as causative agent of bone marrow aplasia with haemorrhagic disease in cattle. The present invention provides novel nucleic acid and protein sequences for diagnostic and therapeutic uses.

The present invention refers to a novel circovirus (CV) as causative agent of bone marrow aplasia with haemorrhagic disease in cattle. The present invention provides novel nucleic acid and protein sequences for diagnostic and therapeutic uses.

### INTRODUCTION

Haemorrhagic diseases in cattle have been associated with a variety of causes including viral infections, hereditary diseases, immune-mediated diseases, bacterial septicaemia, and intoxications. Bleeding tendency and thrombocytopenia are associated with non-cytopathic type 2 bovine viral diarrhoea virus (BVDV) infection (Ellis et al., 1998, Rebhun et al., 1989). A hereditable haemorrhagic diathesis is described for Simmental cattle. This Simmental hereditary thrombopathy is caused by dysfunction of platelets (Steficek, et al., 1993). Immune-mediated thrombocytopenia is known as a rare condition in cows. It may be classified as idiopathic thrombocytopenic purpura or secondary entity (Yeruham, et al., 2003). Examples of bacterial infections include Pasteurella multocida, a well known cause of haemorrhagic septicaemia in calves with petechial and ecchymotic haemorrhages, generalized hyperaemia, and pneumonia as clinical signs (Rhoades, et al., 1967, Rimier, 1978).

Several toxins may be responsible for fatal haemorrhagic diathesis in cattle. Intoxications due to dichlorovinylcysteine (DCVC) in trichloroethylene-extracted soybean oil meal fed to calves (Lock, et al., 1996) and also the antibiotic furazolidone (Hoffmann-Fezer, et al., 1974, Hofmann, et al., 1974) produce fatal aplastic anaemia, marked acellularity of bone marrow and extensive haemorrhages. Ingestion of bracken fern (Pteridium aquilinum) causes acute poisoning in cattle with irreversible bone marrow hypoplasia as well (Maxie and Newman, 2007, Valli, 2007). In addition, intoxications with mycotoxins of Stachybotrys chartarum (atra) have been described in ruminants resulting in pancytopenic disease characterized by profuse haemorrhage and necrosis in many tissues (Harrach, et al., 1983, Valli, 2007).

Chicken infectious anaemia is a disease strongly resembling the haemorrhagic disease in calves reported here. The causative agent is chicken infectious anaemia virus (CIAV). Severe anaemia, severe bone marrow aplasia, atrophy of the thymus and Bursa of Fabricius, and haemorrhages are consistent findings in chicks infected with CIAV (Kuscu and Gurel, 2008, Yuasa, et al., 1979). One-day-old SPF chicks, experimentally inoculated with CIAV, showed a decrease of haematocrit values, became emaciated and depressed with anaemia, particularly between days 12 and 20 post inoculation (Goryo, et al., 1989). CIAV is classified into the family Circoviridae (Todd, et al., 2005). It only infects chicken and is the sole member of the genus Gyrovirus. However, several members of a second genus, Circovirus, have been detected in mammalian and avian species including the porcine circoviruses PCV1 and PCV2. Members of the family Circoviridae are non-enveloped icosahedral particles with a circular single-stranded DNA (ssDNA) genome, 1759 to 2319 nucleotides (nt) in size (Todd, et al., 2005). Viruses in the genus Circovirus possess an ambisense genome organization encoding the replication-associated (Rep) protein from the sense strand (open reading frame [ORF]-V1) and the capsid protein from the complementary sense strand (ORF—C1). Additional small ORFs have been recognized in some of the circoviruses, e.g., ORF3 encoding an apoptosis-inducing protein in PCV2-infected cells (Liu, et al., 2005, Timmusk, et al., 2008). In the noncoding regions, a stem-loop structure is present containing a conserved nonamer sequence and involved in the initiation of the viral genome replication (Steinfeldt, et al., 2001). The molecular biology of circoviruses has been reviewed recently (Mankertz, 2008). With the exception of PCV1, all known circoviruses are pathogens, which cause immunosuppression and damage of the lymphoreticular tissues (Mankertz, 2008, Segales, et al., 2005, Segales and Mateu, 2006, Todd, 2000). PCV2 is a virulent pathogen associated with a number of different syndromes and diseases in pigs such as the post-weaning multisystemic wasting syndrome (PMWS), the porcine respiratory disease complex (PRDC), reproductive failure associated with PCV2 and the porcine dermatitis and nephropathy syndrome (PDNS). However, only lesions typical of PMWS were demonstrated in both colostrum-deprived piglets and conventional pigs by PCV2 inoculation (Ellis, et al., 1999, Kennedy, et al., 2000), whereas the involvement of PCV2 in swine diseases other than PMWS has not been fully investigated (Allan, et al., 2003, Chae, 2005).

Only limited data exist on circovirus infections in cattle. The presence of circoviruses was demonstrated by PCR in lung tissue samples from 6 of 100 cases of bovine respiratory disease and from 4 of 30 aborted fetuses (Nayar, et al., 1999). The genome of this agent, tentatively named bovine circovirus (BCV), was nearly identical to that of PCV2, with 99% overall nucleotide sequence identity. The presence of antibodies reacting with porcine circovirus in sera of humans, mice and cattle has been reported (Tischer, et al., 1995). In another study, however, no antibodies to PCV2 were detected in sera from cattle, sheep, horse and humans (Allan, et al., 2000, Ellis, et al., 2001). Also, a seronegative neonatal calf and six seronegative 6-months-old beef calves that were experimentally infected with PCV2 failed to develop antibodies to the virus (Ellis, et al., 2001).

Since 2007, there have been reports by farmers and veterinarians of an unexplainable haemorrhagic disease in calves all over Germany. 56 calves with spontaneous haemorrhages were presented to the Bavarian Animal Health Service to characterize the lesions and to investigate the aetiology by further laboratory investigations. The disease was observed in young calves of different breeds within their first month of life. Male and female calves were affected likewise. Haemorrhages, particularly in skin, subcutis and gastrointestinal tract, were the major findings. Inflammatory lesions were additional sporadic findings. Histological investigation indicated a severe bone marrow hypo- to aplasia in all animals and lymphocytic depletion in 43% of the affected calves. Blood analysis of 5 animals revealed aplastic pancytopenia. The resulting thrombocytopenia is believed to represent the major pathomechanism of this Haemorrhagic Disease Syndrome (HDS), also referred to as Haemorrhagic Diathesis (HD). Meanwhile, the more common and scientifically accepted name for HDS/HD is Bovine Neonatal Pancytopenia (BNP). The different names and abbreviations may be used interchangeably in the present application. Bacterial infections and infections with bovine viral diarrhoea virus or bluetongue virus were ruled out as cause of the disease. Specific toxins which are known to cause bone marrow aplasia were not detected. Pedigree analysis gave no indication for heredity of the disease.

Using a broad spectrum PCR, the present inventors were able to demonstrate the presence of a circovirus in affected calves. Sequencing of the whole viral genome revealed high similarities with porcine circovirus type 2b (PCV2b). Single bone marrow cells of one calf displayed slight PCV2-antigen immunoreaction.

### SUMMARY OF THE INVENTION

The present invention refers to nucleic acid molecules of a novel circovirus (CV) identified as causative agent of Haemorrhagic Disease Syndrome (HDS), or Haemorrhagic Diathesis (HD), now mainly referred to as Bovine Neonatal Pancytopenia (BNP) in cattle. Further, the invention refers to novel polypeptides encoded by the viral nucleic acid and antibodies directed against these polypeptides. The nucleic acids, polypeptides and antibodies are suitable for diagnostic and therapeutic uses, particularly for the development of vaccines against HD.

In a first aspect, the present invention refers to a circovirus (CV) nucleic acid molecule comprising
(a) the sequence as shown in SEQ ID NO: 1 or a fragment thereof, and/or
(b) the complement of the nucleotide sequence according to (a).

In another aspect, the present invention refers to a circovirus (CV) nucleic acid molecule comprising
(a) the sequence as shown in SEQ ID NO: 7 or a fragment thereof, and/or
(b) the complement of the nucleotide sequence according to (a).

In yet another aspect, the present invention refers to a circovirus (CV) nucleic acid molecule comprising
(a) the sequence as shown in SEQ ID NO: 11 or a fragment thereof, and/or
(b) the complement of the nucleotide sequence according to (a).

The nucleic acid molecule may be a DNA or RNA molecule, which is single- or double-stranded, circular or linear. In some embodiments, the nucleic acid molecule may be present as such or linked to further nucleic acid molecules, e.g. operatively linked with heterologous expression control sequences. The nucleic acid molecule may also be encapsulated in a viral capsid.

The nucleic acid molecule of the present invention may comprise the complete sequence of SEQ ID NO: 1 and/or its complement or a fragment thereof. Likewise, the nucleic acid molecule of the present invention may comprise the complete sequence of SEQ ID NO: 7 or SEQ ID NO: 11 and/or its complement or a fragment thereof. The fragment preferably comprises at least 15, at least 20, at least 25, at least 30 or at least 50 contiguous nucleotides as shown in SEQ ID NO: 1, 7, or 11 or the complement thereof. Preferably, the CV nucleic acid molecule of the present invention differs in at least one nucleotide from related circovirus strains, e.g. circovirus virus strains, whose GeneBank Accession Numbers are indicated in FIG. 6.

The present invention also refers to a nucleic acid molecule
(a) having an identity of at least 90%, at least 94%, at least 96%, at least 98% or at least 99% of the nucleotide sequence as shown in SEQ ID NO: 1,
(b) hybridizing under stringent conditions to the nucleotide sequence as shown in SEQ ID NO:1, or
(c) the complement of (a) or (b).

The present invention also refers to a nucleic acid molecule
(a) having an identity of at least 90%, at least 94%, at least 96%, at least 98% or at least 99% of the nucleotide sequence as shown in SEQ ID NO: 7,
(b) hybridizing under stringent conditions to the nucleotide sequence as shown in SEQ ID NO:7, or
(c) the complement of (a) or (b).

The present invention also refers to a nucleic acid molecule
(a) having an identity of at least 90%, at least 94%, at least 96%, at least 98% or at least 99% of the nucleotide sequence as shown in SEQ ID NO: 11,
(b) hybridizing under stringent conditions to the nucleotide sequence as shown in SEQ ID NO:11, or
(c) the complement of (a) or (b).

The identity of a given nucleic acid molecule to the reference nucleic acid molecule (i.e., for example, SEQ ID NO: 1 or a fragment thereof) may be determined as follows:

I=n/L×100,

wherein I is the identity in percent,
n is the number of identical nucleotides of the given nucleic acid molecule, and the reference, and
L is the length of the sequence overlap of the given nucleic acid molecule and the reference.

Preferably, hybridization under stringent conditions means that after washing for 1 h with 1×SSC-buffer and 0.1% SDS at 50° C., preferably at 55° C., more preferably at 62° C., and most preferably at 68° C., particularly for 1 h in 0.2×SSC and 0.1% SDS at 50° C., preferably at 55° C., more preferably at 62° C., and most preferably at 68° C., a positive hybridization signal is observed. Hybridization protocols are e.g. disclosed in Wahl and Berger (Methods Enzymol. 152 (1987), 399-407) and Kimmel (Methods Enzymol. 152 (1987), 507-511), the content of which is herein incorporated by reference.

A further aspect of the present invention refers to a CV nucleic acid molecule encoding a circovirus polypeptide or a fragment thereof, wherein the nucleic acid molecule comprises
(a) a region of the nucleotide sequence as shown in SEQ ID NO: 1 from
   (i) nucleotide 51-995, (Rep)
   (ii) nucleotide 1034-1735 (Cap)
   (iii) nucleotide 357-671 (ORF3), or
(b) a nucleotide sequence corresponding to the sequence of (a) within the scope of degeneracy of the genetic code, or
(c) a fragment of the nucleotide sequence according to (a) or (b).

Still a further aspect of the present invention refers to a CV nucleic acid molecule encoding a circovirus polypeptide or a fragment thereof, wherein the nucleic acid molecule comprises
(a) a region of the nucleotide sequence as shown in SEQ ID NO: 7 from
   (i) nucleotide 51-995, (Rep)
   (ii) nucleotide 1034-1735 (Cap)
   (iii) nucleotide 357-671 (ORF3), or
(b) a nucleotide sequence corresponding to the sequence of (a) within the scope of degeneracy of the genetic code, or
(c) a fragment of the nucleotide sequence according to (a) or (b).

Still a further aspect of the present invention refers to a CV nucleic acid molecule encoding a circovirus polypeptide or a fragment thereof, wherein the nucleic acid molecule comprises
(a) a region of the nucleotide sequence as shown in SEQ ID NO: 11 from
   (i) nucleotide 51-995, (Rep)
   (ii) nucleotide 1033-1734 (Cap)
   (iii) nucleotide 357-671 (ORF3), or
(b) a nucleotide sequence corresponding to the sequence of (a) within the scope of degeneracy of the genetic code, or
(c) a fragment of the nucleotide sequence according to (a) or (b).

Preferably, the nucleic acid molecule encodes a CV polypeptide selected from Rep (SEQ ID NO: 2), Cap (SEQ ID NO: 3) and ORF3 (SEQ ID NO: 4) or fragments thereof. The nucleic acid molecule may also encode a CV polypeptide selected from Rep (SEQ ID NOs: 8 and 12), Cap (SEQ ID NOs: 9 and 13) and ORF3 (SEQ ID NOs: 10 and 14) or fragments thereof. Fragments of the above indicated CV polypeptides may e.g. comprise at least 6, at least 8, at least 10, at least 20 or at least 30 contiguous amino acids of the amino acid sequences as shown in SEQ ID NO: 2, 3 or 4, or alternatively as shown in SEQ ID NO: 8-10 or 12-14.

Further, the present invention refers to a nucleic acid molecule, which encodes a polypeptide having an identity of at least 90%, at least 92%, at least 94%, at least 96%, at least 98% or at least 99% of any of the amino acid sequences as shown in SEQ ID NO: 2, 3 or 4.

Further, the present invention refers to a nucleic acid molecule, which encodes a polypeptide having an identity of at least 90%, at least 92%, at least 94%, at least 96%, at least 98% or at least 99% of any of the amino acid sequences as shown in SEQ ID NO: 8, 9, 10, 12, 13, or 14.

The degree of identity between a given polypeptide and the reference polypeptides, e.g. of SEQ ID NO: 2, 3 or 4, may be determined as indicated for nucleic acid molecules above.

The nucleic acid molecule of the present invention may be in operative linkage with a heterologous expression control sequence, e.g. an expression control sequence allowing expression in a suitable host cell. Examples of heterologous expression control sequences for expressing the nucleic acid sequence of the present invention, e.g. prokaryotic or eukaryotic including mammalian expression control sequences unknown to the skilled person and e.g. disclosed in Sambrook et al., Molecular Cloning, A Laboratory Manual, Cold Spring Harbour Press, and Ausubel et al. (1989), Current Protocols in Molecular Biology, John Wiley and Sons, the content of which is herein incorporated by reference.

The present invention also encompasses a non-human host cell, e.g. a prokaryotic or eukaryotic host cell, e.g. a yeast, insect or mammalian host cell, which is transformed or transfected with a nucleic acid molecule as indicated above. Transformation or transfection of host cells with nucleic acid molecules e.g. located on a vector, e.g. a viral vector or a plasmid are well known to the skilled person and e.g. described in Sambrook et al. (supra) or Ausubel et al. (supra).

Still a further aspect of the present invention is a circovirus (CV) polypeptide encoded by a nucleic acid molecule as described above. A CV polypeptide may comprise
(a) an amino acid sequence selected from
   (i) amino acid sequence SEQ ID NO: 2 (Rep)
   (ii) amino acid sequence SEQ ID NO: 3 (Cap)
   (iii) amino acid sequence SEQ ID NO: 4 (ORF3), or
(b) a fragment thereof.

Still a further aspect of the present invention is a circovirus (CV) polypeptide encoded by a nucleic acid molecule as described above. A CV polypeptide may comprise
(a) an amino acid sequence selected from
   (i) amino acid sequence SEQ ID NO: 8 and 12 (Rep)
   (ii) amino acid sequence SEQ ID NO: 9 and 13 (Cap)
   (iii) amino acid sequence SEQ ID NO: 10 and 14 (ORF3), or
(b) a fragment thereof.

The present invention comprises CV polypeptides or fragments thereof, which comprise at least 6, at least 8, at least 10, at least 20 or at least 30 contiguous amino acids of the amino acid sequences as shown in SEQ ID NO: 2, 3 or 4 or, alternatively, as shown in SEQ ID NO: 8-10 or 12-14. Preferably, a CV polypeptide of the present invention differs in at least 1 amino acid from related circovirus strains, e.g. circovirus strains, whose GeneBank Accession Numbers are indicated in FIG. 6.

The invention also refers to polypeptides having an identity of at least 90%, at least 92%, at least 94%, at least 96%, at least 98% or at least 99% of any of the amino acid sequences as shown in SEQ ID NO: 2, 3 or 4.

The invention also refers to polypeptides having an identity of at least 90%, at least 92%, at least 94%, at least 96%, at least 98% or at least 99% of any of the amino acid sequences as shown in SEQ ID NO: 8, 9, 10, 12, 13, or 14.

Still a further aspect of the present invention is an antibody directed against a polypeptide as described above or an antigen-binding fragment of such antibody.

Methods of generating antibodies e.g. polyclonal or monoclonal antibodies are well known in the art. For example, various mammalian hosts, e.g. mice or rabbits may be immunized by injection of a polypeptide of the invention, which has immunogenic properties. If desired, the polypeptide of the present invention may be coupled to a carrier such as keyhole limpet hemocyanin (KLH). From the immunized host polyclonal antibodies or antibody-producing cells may be obtained by well known methods.

Monoclonal antibodies directed against the polypeptides of the invention may be prepared by known techniques, e.g. the B-cell hybridoma technique (Köhler et al., Nature 256; (1975) 495-497), the content of which is herein incorporated by reference or related techniques.

The present invention also encompasses chimeric, humanized or human antibodies or antigen-binding fragments of such antibodies, which may be obtained by known techniques.

Still a further aspect of the present invention is a circovirus comprising a nucleic acid molecule as described above. The virus may be an active virus. Alternatively, the virus my be an inactivated virus or an attenuated virus. Inactivation and attenuation may be effected as described in detail below.

The nucleic acid molecules, polypeptides, viruses and antibodies of the present invention may be used as a diagnostic or pharmaceutic agent, e.g. for the diagnosis or prevention and/or treatment of HD in mammals, particularly in cattle and more particularly in calves.

In diagnostic embodiments, the nucleic acid molecule or the polypeptide may carry a reporter group, e.g. any reporter group suitable for use in diagnostic methods, e.g. fluorescent groups, luminescent groups, dyes, enzymes, haptens or biotin.

Particularly, for diagnostic embodiments, the term “nucleic acid molecule” as used in the present application also encompasses nucleic acid analogues such as peptide nucleic acid (PNA) locked nucleic acids (LNA) or other types of nucleic acid analogues known in the art.

Thus, a further aspect of the present invention is a diagnostic composition comprising a nucleic acid molecule, a polypeptide, a virus or an antibody as described above together with an acceptable carrier.

A diagnostic composition may be used in a method for diagnosing HD, particularly in cattle, wherein a sample from the subject to be diagnosed is contacted with a diagnostic composition as described above, such that the presence and/or amount of CV, particularly strain PCV2-Ha08, PCV2-Ha09 or PCV2-Ha10 or of antibodies against CV, particularly strain PCV2-Ha08, PCV2-Ha09 or PCV2-Ha10 in that sample is determined. The sample may be a body fluid sample, e.g. blood, serum, plasma, saliva, sputum, or lymph fluid, or a tissue sample, e.g. from liver, lung, bone marrow or lymphatic tissue.

In one embodiment, the diagnostic method of the invention may encompass determination of CV nucleic acid molecules, e.g. in nucleic acid based assays, which may involve hybridization and nucleic acid amplification techniques such as PCR. Further, the diagnostic method of the invention encompasses determination of CV polypeptides, e.g. in immunoassays using antibodies of the invention as diagnostic reagents and determining the presence of immune complexes of CV polypeptides and detections antibodies. On the other hand, the diagnostic method of the invention may encompass the determination of anti-CV antibodies in the sample, e.g. using CV polypeptides as described above as detection antigens.

Still a further embodiment of the present invention is the use of the above nucleic acid molecules, polypeptides, viruses and antibodies for therapeutic applications, particularly for the treatment and/or prevention of HD in mammalian organisms, particularly in cattle.

Thus, the present invention also encompasses a composition for therapeutic use comprising the nucleic acid molecule, the polypeptide, the antibody or the virus as described above together with a pharmaceutically acceptable carrier, diluent and/or adjuvant. In a preferred embodiment, the composition is a vaccine or an immunogenic composition, e.g. a nucleic acid based vaccine or immunogenic composition, or a polypeptide based vaccine or immunogenic composition, a virus based vaccine or immunogenic composition or an antibody based vaccine. In an especially preferred embodiment, the composition is a polypeptide based vaccine or immunogenic composition and comprises a CV polypeptide capable of eliciting an immune response in a subject together with a pharmaceutically acceptable carrier, diluent and/or adjuvant. In another especially preferred embodiment, the composition is a virus based vaccine or immunogenic composition and comprises a circovirus capable of eliciting an immune response in a subject together with a pharmaceutically acceptable carrier, diluent and/or adjuvant.

The invention also encompasses a method of preventing or treating HD, particularly in mammalian subjects, such as cattle, wherein a therapeutic composition as described above is administered to a subject in need thereof in an effective amount.

For therapeutic applications, nucleic acid molecules may either be used in form of nucleic acid based vaccines or immunogenic compositions or as nucleic acid effector molecules, such as antisense molecules, or molecules capable of RNA interference. Polypeptides or viruses of the present invention may be used in therapeutic applications for the manufacture of polypeptide- or virus-based vaccines or immunogenic compositions as described above. Antibodies may be used in therapeutic applications for the treatment of already existing CV infections.

### DETAILED DESCRIPTION OF THE INVENTION

The following definitions may be applied to terms employed in the description of embodiments of the invention. The following definitions supercede any contradictory definitions contained in each individual reference incorporated herein by reference.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The term “adjuvant”, as used herein, refers to any substance which serves as a non-specific stimulator of the immune response. Suitable adjuvants include, but are not limited to, the RIBI adjuvant system (Ribi Inc.), alum, aluminum hydroxide gel, oil-in water emulsions, water-in-oil emulsions such as, e.g., Freund's complete and incomplete adjuvants, Block co-polymer (CytRx, Atlanta Ga.), SAF-M (Chiron, Emeryville Calif.), AMPHIGEN® adjuvant, ionic polysaccharides, saponin, Quil A, QS-21 (Cambridge Biotech Inc., Cambridge Mass.), GPI-0100 (Galenica Pharmaceuticals, Inc., Birmingham, Ala.) or other saponin fractions, Procision-ATM (an adjuvant that comprises an admixture of Quil A, AMPHIGEN® and cholesterol), monophosphoryl lipid A, Avridine lipid-amine adjuvant, heat-labile enterotoxin from E. coli (recombi-nant or otherwise), cholera toxin, or muramyl dipeptide, among many others known to those skilled in the art.

Reference to an “ionic polysaccharide” should be understood as a reference to any positively or negatively charged polysaccharide or derivative or chemical equivalent thereof. Said ionic polysaccharide may be in soluble or insoluble form. Preferably said ionic polysaccharide is an ionic dextran. Even more preferably said ionic dextran is DEAE-dextran, dextran sulphate or QAE-dextran. Most preferably, said ionic dextran is DEAE dextran. Preferably, the dextran component of said ionic dextran exhibits a molecular weight in the range 250,000 to 4,000,000 Da and even more preferably 500,000 to 1,500,00 Da.

The adjuvant properties of saponin have been long known, as has its ability to increase antibody titres to immunogens. As used herein, the term “saponin” refers to a group of surface-active glycosides of plant origin composed of a hydrophilic region (usually several sugar chains) in association with a hydrophobic region of either steroid or triterpenoid structure. Although saponin is available from a number of diverse sources, saponins with useful adjuvant activity have been derived from the South American tree Quillaja saponaria (Molina). Saponin from this source was used to isolate a “homogeneous” fraction denoted “Quil A” (Dalsgaard, 1974).

Dose-site reactivity is a major concern for both the veterinary and human use of Quil A in vaccine preparations. One way to avoid this toxicity of Quil A is the use of immunostimulating complexes (known as Iscoms™, an abbreviation for Immuno Stimulating COMplexes). This is primarily because Quil A is less reactive when incorporated into immunostimulating complexes, because its association with cholesterol in the complex reduces its ability to bind to cholesterol in cell membranes and hence its cell lytic effects. In addition, a lesser amount of Quil A is required to generate a similar level of adjuvant effect.

The immunomodulatory properties of the Quil A saponins and the additional benefits to be derived from these saponins when they are incorporated into an immunostimulating complex have been described in various publications, e.g. Cox and Coulter, 1992 (Cox, J. C. and Coulter, A. R., “Advances in Adjuvant Technology and Application”, in Animal Parasite Control Utilizing Biotechnology, Chapter 4, Ed. Yong, W. K., CRC Press (1992)); Dalsgaard, 1974; Morein et al., Australian Patent Specifications Nos. 558258, 589915, 590904 and 632067.

The amounts and concentrations of adjuvants and additives useful in the context of the present invention can readily be determined by the skilled artisan. In one embodiment, the present invention contemplates immunogenic compositions and vaccines comprising from about 50 μg to about 2000 μg of adjuvant. In another embodiment adjuvant is included in an amount from about 100 μg to about 1500 μg, or from about 250 μg to about 1000 μg, or from about 350 μg to about 750 μg. In another embodiment, adjuvant is included in an amount of about 500 μg/2 ml dose of the immunogenic composition or vaccine.

The term “amino acid,” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, carboxyglutamate, and O-phosphoserine. Stereoisomers (e.g., D-amino acids) of the twenty conventional amino acids, unnatural amino acids such as α and α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids.

Amino acid analogs refer to compounds that have the same basic chemical structure as a naturally occurring amino acid, i.e., a carbon that is bound to a hydrogen, a carboxyl group, an amino group, and an R group. Exemplary amino acid analogs include, for example, homoserine, norleucine, methionine sulfoxide, and methionine methyl sulfonium. Such analogs have modified R groups (e.g., norleucine) or modified peptide backbones, but retain the same essential chemical structure as a naturally occurring amino acid. Amino acid mimetics refer to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission.

The term “antibody” or “antibodies”, as used herein, refers to an immunoglobulin molecule able to bind to an antigen by means of recognition of an epitope. Antibodies can be a polyclonal mixture or monoclonal. Antibodies can be intact immunoglobulins derived from natural sources or from recombinant sources, or can be immunoreactive portions of intact immunoglobulins. Antibodies can exist in a variety of forms including, for example, as, Fv, Fab′, F(ab′)₂, as well as in single chains.

The term “antigen” as used herein refers to a molecule that contains one or more epitopes (linear, conformational or both) that upon exposure to a subject will induce an immune response that is specific for that antigen. The term “antigen” as used herein can refer to attenuated, inactivated or modified live bacteria, viruses, fungi, parasites or other microbes. The term “antigen” as used herein can also refer to a subunit antigen, which is separate and discrete from a whole organism with which the antigen is associated in nature. The term “antigen” also as used herein can also refer to antibodies, such as anti-idiotype antibodies or fragments thereof, and to synthetic peptide mimotopes that can mimic an antigen or antigenic determinant (epitope). The term “antigen” as used herein can also refer to an oligonucleotide or polynucleotide that expresses an antigen or antigenic determinant in vivo, such as in DNA immunization applications.

The circovirus of the present invention can be “attenuated” or “inactivated” prior to use in a vaccine. Methods of attenuation and inactivation are well known to those skilled in the art. Methods for attenuation include, but are not limited to, serial passage in cell culture on a suitable cell line, ultraviolet irradiation, and chemical mutagenesis. Methods for inactivation include, but are not limited to, treatment with formalin, betapropriolactone (BPL) or binary ethyleneimine (BEI), or other methods known to those skilled in the art.

Inactivation by formalin can be performed by mixing the virus suspension with 37% formaldehyde to a final formaldehyde concentration of 0.05%. The virus-formaldehyde mixture is mixed by constant stirring for approximately 24 hours at room temperature. The inactivated virus mixture is then tested for residual live virus by assaying for growth in a suitable cell line.

Inactivation by BEI can be performed by mixing the virus suspension of the present invention with 0.1 M BEI (2-bromo-ethylamine in 0.175 N NaOH) to a final BEI concentration of 1 mM. The virus-BEI mixture is mixed by constant stirring for approximately 48 hours at room temperature, followed by the addition of 1.0 M sodium thiosulfate to a final concentration of 0.1 mM. Mixing is continued for an additional two hours. The inactivated virus mixture is tested for residual live virus by assaying for growth on a suitable cell line.

The term “cell line” or “host cell”, as used herein means a prokaryotic or eukaryotic cell in which a virus can replicate and/or be maintained.

The term “immunogenic composition” as used herein means a composition capable of inducing an immune or antigenic response in a subject.

The term “pharmaceutically-acceptable carrier” as used herein refers to substances, which are within the scope of sound medical judgment, suitable for use in contact with the tissues of humans or animals without undue toxicity, irritation, allergic response, and the like, commensurate with a reasonable benefit-to-risk ratio, and effective for their intended use. Vaccines of the present invention can include one or more pharmaceutically-acceptable carriers, such as all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others known to those skilled in the art. Stabilizers include albumin, among others known to the skilled artisan. Preservatives include merthiolate, among others known to the skilled artisan.

The term “polynucleotide or nucleic acid molecule” as used herein means an organic polymer molecule composed of nucleotide monomers covalently bonded in a chain. DNA (deoxyribonucleic acid) and RNA (ribonucleic acid) are examples of polynucleotides with distinct biological function.

The terms “prevent”, “preventing” or “prevention”, and the like, as used herein, mean to inhibit the replication of a microorganism, to inhibit transmission of a microorganism, or to inhibit a microorganism from establishing itself in its host. The terms and the like as used herein can also mean to inhibit or block one or more signs or symptoms of infection.

The term “therapeutic agent” as used herein means a microorganism (or parts thereof), or a subunit antigen, or polypeptides, or polynucleotide molecules, and combinations thereof, which elicits an immune response in the subject to which it is administered. The immune response can comprise, without limitation, induction of cellular and/or humoral immunity.

The terms “treat”, “treating” or “treatment”, and the like, as used herein mean to reduce or eliminate an infection by a microorganism. The terms and the like as used herein can also mean to reduce the replication of a microorganism, to reduce the transmission of a microorganism, or to reduce the ability of a microorganism to establish itself in its host. The terms and the like as used herein can also mean to reduce, ameliorate, or eliminate one or more signs or symptoms of infection by a microorganism, or accelerate the recovery from infection by a microorganism.

The terms “vaccine” and “vaccine composition,” as used herein, mean a composition which prevents or reduces an infection, or which prevents or reduces one or more signs or symptoms of infection. The protective effects of a vaccine composition against a pathogen are normally achieved by inducing in the subject an immune response, either a cell-mediated or a humoral immune response or a combination of both. Generally speaking, abolished or reduced incidences of infection, amelioration of the signs or symptoms, or accelerated elimination of the microorganism from the infected subjects are indicative of the protective effects of a vaccine composition. The vaccine compositions of the present invention provide protective effects against infections caused by circovirus (CV).

The following Figures and Examples described are provided to aid those skilled in the art in practicing the present invention. Even so, this description should not be construed to unduly limit the present invention as modifications and variations in the embodiments discussed herein can be made by those of ordinary skill in the art without departing from the spirit or scope of the present inventive discovery.

### FIGURES

FIG. 1. Localizations of haemorrhages in diseased calves. A: Focal acute bleedings in the skin of the head. Small tufts of hair stuck together by dried blood. B: Petechial and ecchymotic haemorrhages in the mucosa of the lower lip and gingiva. C: With the exception of bleedings associated with injection sites and ear tagging there was no evidence of traumatic skin injuries. D: Moderate focal haemorrhages in the mesenterium of small and large intestine. The segmental dark red discoloration of the small intestine is due to severe intraluminal bleedings. E: Subcutis of the carpus. Subcutaneous haemorrhages are seen most often over bone protrusions and mechanical strained parts of the body.

FIG. 2. Frequency of additional findings in calves with pancytopenia and haemorrhagic disease (BNP). Some animals showed several additional lesions. Inflammations of different organs were found frequently, but 30% of the investigated animals had no further lesions. GIT: gastrointestinal tract.

FIG. 3. Histological investigation of bone marrow (sternal bone) after decalcification, HE stain, 100× magnification. A: Normal bone marrow of a three-weeks-old calf with haematopoietic tissue including several megakaryocytes (arrows). B: Bone marrow of an affected calf with severe loss of haematopoietic tissue. Only stromal fibroblasts and fat cells remained.

FIG. 4. Detection of circovirus DNA in samples from calves with haemorrhagic disease. A nested broad-spectrum PCR was performed using DNA extracted from bone marrow (lanes 3, 5, 9-12), blood (lanes 4 and 8), liver (lane 6) or kidney (lane 7) of calves with numbers indicated above the lanes. Neg: negative isolation control; pos: positive PCR control; M: molecular mass markers, with sizes indicated left in bp. The secondary PCR products with a size of approximately 350 bp had been separated on an ethidium-bromide stained agarose gel.

FIG. 5. Immunohistochemistry for detection of PCV2-specific antigen, sternum, calf No. 1. Single bone marrow cells showed mild to moderate finely granular cytoplasmic staining. Bar 100 μm.

FIG. 6. Phylogenetic relationship of the circovirus PCV2-Ha08 detected in a German calf with porcine circovirus type 2 strains. The phylogenetic tree was established on the basis of the complete nucleotide sequences of the reference strains PCV2a, PCV2b and PCV2c (bold face), the Canadian bovine circovirus (BCV) and ten circoviruses which turned out to be most closely related to PCV2-Ha08 by a BLAST search. PCV2-Ha08 is marked with an arrow. The GenBank accession numbers of the sequences are shown in brackets. The tree is scaled in nucleotide substitution units.

FIG. 7: Nucleotide and amino acid sequence of PCV2 strain Ha08 according to unpublished Gen Bank entry Accession No. FJ804417.

FIG. 8: Nucleotide and amino acid sequence of PCV-2 strain Ha09 according to unpublished Gen Bank entry Accession No. HQ231329.

FIG. 9: Nucleotide and amino acid sequence of PCV-2 strain Ha10 according to unpublished Gen Bank entry Accession No. HQ231328.

FIG. 10: Analysis of sera in a prototype species-independent PCV2 Enzyme-Linked Immunosorbent Assay (ELISA). Solid lines represent porcine PCV2-negative sera, dashed lines represent porcine PCV2-positive sera, and dotted lines represent bovine BNP sera.

### EXAMPLES

### Materials and Methods

### Case History

Between October 2007 and May 2009, 56 calves with haemorrhagic disease, originating from 45 dairy cattle farms in Bavaria, Germany, were presented for necropsy. Medical records were reviewed for age, sex, and breed. Owners were asked for previous diseases and previous medical treatment of calves, feeding of calves, contamination of forage with mould or bracken fern, and use of rodenticides.

HDS (BNP) cases and animals of the control group are numbered according to Table 1. Animals of the control group are specified as those in the text.

Eight calves, sent for pathological examination for other reasons than haemorrhagic disease, were included as controls for the circovirus-specific PCR; they are listed in Table 1. Control No. 1 belonged to the same livestock as two cases with haemorrhagic disease (Nos. 11 and 15) and died shortly after birth for unknown reasons. No infectious agent was detectable in this case. Seven calves, included in the control group because of their age, suffered from severe polyarthritis or severe enteritis and died within the first month of life. None of the control animals showed any signs of bone marrow depletion.

### Histopathology

All animals underwent necropsy examination, and a standard series of tissues including bone marrow of femur and sternal bone, lung, liver, kidney, spleen, and lymph nodes were collected for histopathology. Additional samples were collected depending on further pathological findings, as required. Specimens of organ tissue were fixed in 10% buffered formalin. Specimens of sternal bone marrow were decalcified overnight in Ossa Fixona® (Waldeck, Munster, Germany). Following processing for paraffin embedding, 4-μm-thick sections were cut and stained with haematoxylin and eosin (HE).

### Immunohistochemistry

Immunohistochemistry (IHC) was performed on 4-mm sections mounted on Superfrost® Plus glass slides. A mouse monoclonal antibody, 36A9, directed against the VP2 protein (ORF2) of PCV2 (Ingenasa, Madrid, Spain) was applied to tissue sections of bone marrow, spleen, and lymph node of 2 affected calves. Reactivity of the antibody was assessed in each run on sections of lymph node and Payers Patches collected from a pig with confirmed PCV2 infection based upon immunohistochemistry and PCR analysis. Prestain treatment included xylene washes to deparaffinise the sections and serial graded ethanol washes for rehydration followed by treatment with 3% hydrogen peroxide to quench endogenous tissue peroxidase activity. Staining was formed using the Histostain®-Plus Bulk Kit and the chromogen reagent AEC Single Solution (Invitrogen™, Camarillo, Calif., USA) according to the manufacturer's instructions. Finally, sections were counterstained with Mayer's haematoxylin.

Slides classified as PCV2 positive showed an intracytoplasmatic, bright red signal in a granular pattern.

### Haematology

EDTA blood samples were available from 5 cases (Nos. 2, 53-56), and blood analysis was performed within 48 hours after collection. Complete blood count was calculated including white blood cell count, platelet count, haemoglobin level and parameters of red blood cells using the CELL-DYN® 3500 (Abbott, Wiesbaden, Germany) equipment. The microscopic method was used to assess the number of platelets on a haemocytometer slide

### Toxicology

The following samples were tested for specific toxins: Urine and blood samples of cases Nos. 21 and 22 were analyzed with specific methods to detect dichlorovinylcysteine (DCVC) and its metabolites. Gas chromatography-mass spectrometry (GC-MS) method was used for the detection of volatile organic compounds, coumarine derivatives and chemotherapeutics such as sulphonamides in urine samples of case No. 25 and renal tissue of case No. 8. Samples of urine and liver of three cases (Nos. 23, 34, and 36) were tested for pharmaceutical drugs using GC-MS method and high-performance liquid chromatography (HPLC) method. Forage samples (silage, hay, soybean extraction meal, and straw) were collected from a farm with two affected cases (calves Nos. 1 and 2). A sample of straw was suspicious due to greyish discoloration and mouldy smell. Mycotoxicological investigations as well as a cytotoxicity assay were performed with regard to Aflatoxin B1 and toxins of Stachybotrys chartarum. Attempts to demonstrate the presence of mycotoxins (Fumitremorgen C, Verrucologen, Aflatoxin B1, Fumagillin, Gliotoxin, Verrucarol NH4+, Deoxynivalenol, Nivalenol, Zearalenon, Satratoxin G, Satratoxin H, Verrucarin A, Roridin A, Roridin L, Satratoxin F, and Verrucarin J) were made using LC-MS/MS analysis as published recently (Gottschalk, et al., 2008). Cytotoxicity (MTT) assays were performed according to the method of Reubel et al. (1987).

### Microbiological Culture

A standard set of organs (lung, liver, spleen, kidney, and small intestine) of all animals in the study and the control group as well as additional samples depending on pathological findings were examined for the presence of bacteria. Each sample was investigated by inoculating Columbia blood agar with 5% defibrinated sheep blood and Water-blue-metachrome-yellow lactose agar. Brain-heart-infusion-agar and chocolate-agar were used for detection of microaerophilic germs in lungs. For anaerobic examination, Zeissler agar was used. Salmonella were isolated in Rappaport-Vassilioadis medium after pre-enrichment in buffered peptone water and Xylose lysine desoxycholate agar.

### Virology

Renal and thyroid tissues of all affected animals were tested for the presence of BVDV by direct immunofluorescence assay using a diagnostic kit (Bio-X Diagnostics, Jemelle, Belgium) according to the manufacturer's instructions. For isolation of BVDV, monolayers of bovine KOP—R cells (RIE 244, CCLV Federal Research Centre for Virus Diseases of Animals, Island of Riems, Germany) were inoculated with organ homogenates. The cells were screened daily for cytopathic changes. After a second cell culture passage, the cells were examined by direct immunofluorescence assay as described and by an indirect ELISA for the detection of BVDV-specific antigens (SERELISA BVD p80 Ag Mono Indirect, Synbiotics, Lyon, France). For the demonstration of BVDV-specific nt sequences, RNA was isolated from tissue samples using the RNeasy Mini Kit (Qiagen, Hilden, Germany), and a commercial real-time RT-PCR protocol (Virotype BVDV Kit; Labor Diagnostik Leipzig, Leipzig, Germany) was applied according to the manufacturer's instructions.

For detection of BTV-specific sequences, a real-time RT-PCR protocol covering all 24 BTV serotypes (Toussaint, et al., 2007) was carried out with RNA isolated from spleen tissue of all affected calves.

Out of a total of 56 calves in the study group, 25 were randomly selected (Cases No. 1, 2, 4-13, 15-22, 31, 34, 41, 42, 45) for the detection of mammalian and avian circoviruses including PCV2; all calves in the control group, (designated as control No. 1, control No. 2, etc.) were also investigated. DNA was extracted from tissues including blood, bone marrow, spleen, thymus, kidney, and liver using the High Pure PCR Template Preparation Kit (Roche, Mannheim, Germany), and a nested broad-spectrum PCR protocol was applied as recently described (Halami, et al., 2008). A further PCR protocol, routinely applied for specific detection of PCV2, was performed according to Bogner et al. (2005). Precautions were made to exclude laboratory DNA contamination during PCR analysis. DNA isolation, preparation of PCR mastermix and analysis of PCR products were performed in separate rooms with different sets of pipettes and the exclusive use of filter tips. Each set of reactions was screened for contamination using a negative reagent control and a negative DNA isolation control. The laboratory has never been used for routine PCR diagnostics for PCV2 infection prior to the commencement of this investigation.

### Amplification of the Whole Circovirus Genome

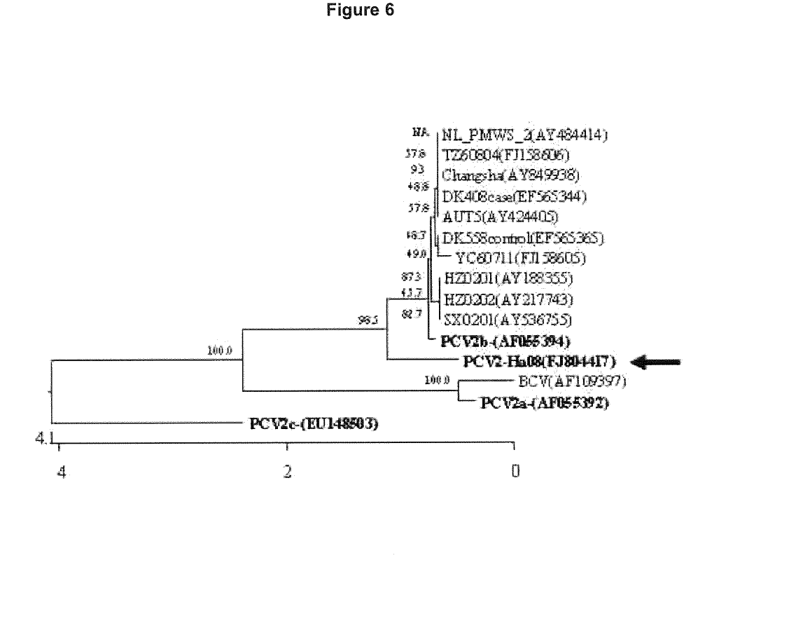

The complete genome of the detected circovirus was amplified by PCR using a pair of inverse primers (5′-AGC TCC ACA CTC GAT CAG TAAG-3′ (SEQ ID NO: 5) and 5′-CCT AGA TCT CAG GGA CAA CGG AG-3′ (SEQ ID NO: 6)), designed according to the sequence amplified by the nested broadspectrum PCR. Amplification was performed using the High Fidelity PCR Enzyme Mix (Fermentas, St. Leon-Rot, Germany) with the following cycling conditions: initial denaturation at 95° C. for 5 min followed by 35 cycles of 95° C. for 30 sec, 58° C. for 30 sec and 70° C. for 4 min, and a final extension at 70° C. of 10 min. DNA sequencing and phylogenetic analysis The PCR products were cloned using the GeneJET. PCR Cloning Kit (Fermentas, St. Leon-Rot, Germany). The insert of the plasmids was sequenced using the primers pJet1 forward and pJet1 reverse (Fermentas, St. Leon-Roth, Germany) or specific primers in an ABI Prism device (Applied Biosystems). The complete genome sequence of the detected circovirus was reassembled from the sequence fragments using the EditSeq module of the Lasergene DNASTAR software package (DNASTAR, Inc., Madison, Wis., USA) and subsequently deposited in the GenBank database with the accession no. FJ804417. Sequence similarity searches were performed using the BLAST 2.2.14 search facility. Sequence alignments and construction of phylogenetic trees were carried out with the CLUSTAL W method (Thompson, et al., 1994) using the MegAlign module of the above-mentioned software package. The strain designations and GenBank accession numbers are presented in FIG. 6.

### Pedigree Analysis

All calves and their parents were identified and traced by their ear tags. The pedigree of all cases was constructed from the pedigree that is used for the joint breeding evaluation of Germany and Austria. The graphical presentation of the pedigree was performed with the Pedigraph™ software and sires occurring more than once were identified.

### Results

86% of the examined calves were Simmental cattle (n=48), 4% were Holstein Friesian cattle (n=2) and 11% were of mixed or unknown breed (n=6). Age at time of death ranged from 7 to 32 days (17 days in average). 85% of the calves fell ill in the second to third week of life. Male and female calves were affected in equal shares. Retrospective analysis of clinical history revealed that the calves were healthy at time of birth and during the first days post partum. Owners and attending veterinarians reported spontaneous transcutaneous bleedings without any obvious injury and haemorrhages in several mucosal surfaces as well as excessive bleeding associated with trauma or standard management procedures such as ear tagging or injections. Sometimes, additional signs such as fever, diarrhoea, or dyspnea were recorded. Haemorrhages seemed to emerge only in single or a couple of calves of a farm at irregular intervals. Medical treatment was unsuccessful. Most calves died within days (n=50) or had to be euthanized (n=6) in consequence of the blood loss.

All calves had received colostrum in the first days of life. Thereafter, most farmers fed whole milk from cows of their own. In general, calves remained untreated until first signs of haemorrhages emerged. Some calves received preventive medication, or because of acute diarrhoea, some were treated with halofuginone against Cryptosporidia. As in Germany bracken fern is a component of pastures only in a minor degree, any problems due to bracken fern contamination had not been reported. Rodenticides were used on the farms, but owners excluded a possible ingestion by cows or calves. Only one of the farmers mentioned to have experienced health problems in cattle due to mouldy forage.

### Pedigree Analysis

The pedigree of all calves was constructed. The parentage of the calves was diverse and indicated no monogenic (recessive or dominant) genetic cause of disease. Even though some sires were represented several times, the number of calves was too small to obtain meaningful results from this analysis.

### Gross Pathology

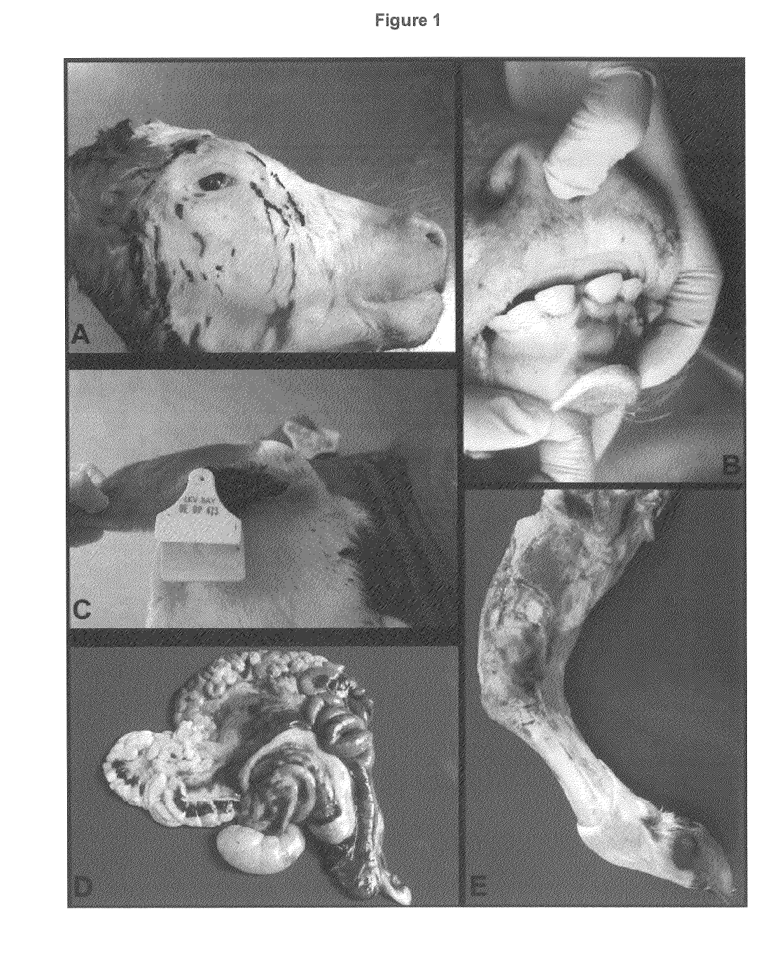

At necropsy, the carcasses of the 56 calves in the study group were in good nutritional state with bodyweights between 38 and 72 kg, depending on age (53 kg in average). In most of the animals, the abomasum contained coagulated milk, and some straw was found in the rumen. There was no indication of an uptake of toxic plants such as bracken fern. Predominant pathomorphological findings in all 56 cases were severe acute haemorrhages in various organs and tissues. 88% of the animals showed multifocal petechial to ecchymotic haemorrhages in skin and subcutis. Haemorrhages in the serosal and mucosal surfaces of the gastrointestinal tract, in some cases with severe melena, occurred very frequently (98%). Furthermore, haemorrhages in the heart, the meninges, and skeletal muscle were common findings (up to 84%). Examples of haemorrhages are shown in FIG. 1. The bone marrow of long bones and sternum was pale red. Depending on duration and intensity of bleedings, the carcasses appeared anaemic.

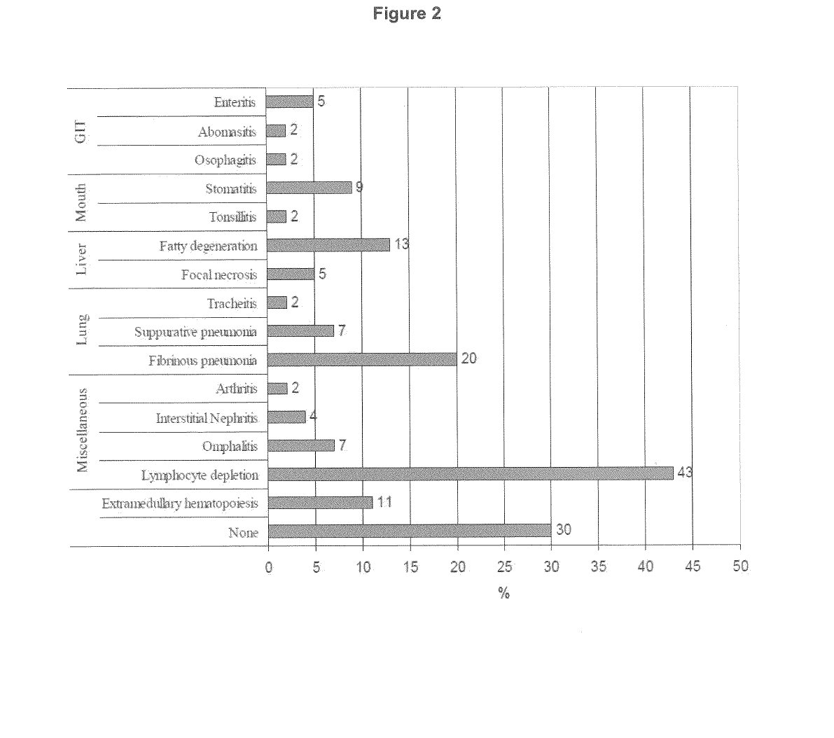

Inflammatory lesions were additional sporadic findings. Fibrinous or suppurative pneumonia (in total 27%) and focal ulzerative to necrotizing inflammation in the oral cavity (in total 11%) were observed most frequently. Additional pathological and histological findings are listed in FIG. 2.

### Histopathology

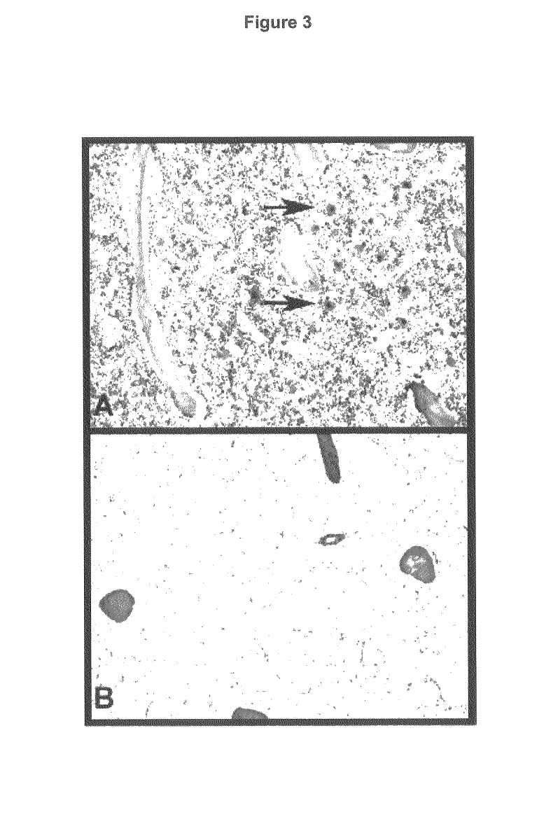

The major histopathological finding was a marked hypo- to acellularity of haemopoietic tissue in the bone marrow in each of the 56 animals (FIG. 3). All haematopoietic lineages were affected in the same way. In some cases, small islands of haematopoietic tissue remained. Occasionally, focal degeneration and apoptosis of precursor cells was present in these locations. Spaces between stromal cells were hyperaemic or filled with homogeneous eosinophilic material, or haematopoietic tissue was replaced by fat tissue. Only five of the 56 cases (9%) showed evidence of extramedullary haematopoiesis. Bleeding sites showed no further changes which would explain the bleeding tendency due to previous tissue damages such as vasculitis, inflammatory reactions, or tissue disruption. In 43% of the cases (n=24), lesions in lymphatic tissues became evident as an increased number of apoptotic lymphocytes in lymphoid follicles or low cellularity of spleen and lymph nodes with small follicles. These changes were summarized as lymphocytic depletion. An occasional and infrequent finding was the presence of few multinucleated giant cells in lymphatic tissue (n=2). The cellular inflammatory reaction in some ulcerative lesions of the oral cavity was mainly composed of mononuclear cells with strikingly few neutrophils. Likewise, in some cases of fibrinous pneumonia, the inflammatory exudate consisted of large quantities of fibrin with very few neutrophils. Additional histological findings are also listed in FIG. 2. There was no evidence of jaundice or haemolysis. No inclusion bodies were recognized in haematopoietic or lymphatic tissues.

### Haematology

EDTA blood was available from 5 cases (Nos. 2, 53-56). Blood analysis revealed severe thrombocytopenia (12.5−82×103 cells/μl), moderate to severe leucopenia (285-1.470 cells/μl), and moderate relative lymphocytosis (68-96%) in all 5 cases. Additionally, 4 of theses cases showed a marked decrease of neutrophil granulocytes (granulocytopenia, 1-4%). 3 cases were anaemic. The haematocrit of 2 cases still ranged in physiological limits. Detailed haematologic results are presented in Table 2.

### Toxicology

Toxicological screening of urine and renal tissue of cases Nos. 8 and 25 indicated no evidence for uptake of substances such as trichloroethylene, anticoagulants or sulfonamides. The antibiotic furazolidone was not detectable in samples of urine and liver of cases Nos. 23, 34, and 36 using HPLC method. However, metamizol was found in cases Nos. 23 and 34 and a combination of sulfamethazin and trimethoprim was found in case No. 36. These results were interpreted to be the result of therapeutical administration shortly before death. In addition, analysis of urine and blood samples collected from 2 cases using specific methods for detection of DCVC and its metabolite N-acetyl-DCVC yielded negative results.

The condition of straw collected from one farm suggested a possible contamination with mould, however, no mycotoxins were detected. The cytotoxicity assay also showed negative results.

### Microbiological Culture

All cases of calves with haemorrhagic disease were tested for the presence of potentially pathogenic bacteria. In some cases, more than one agent was detected. E. coli (n=29) was detected most often, followed by C. perfringens (n=14) in intestine and other organs. P. multocida (n=3) and P. aeruginosa (n=3) were found in few cases. M. haemolytica, Pseudomonas spp., Staphylococci, Nocardia spp. and Salmonella enterica. were found only in single animals (n=1 each). In 16 cases no bacterial pathogens were detected.

26 cases of calves with haemorrhagic disease showed additional inflammatory lesions (FIG. 2). Pneumonia was most frequently observed (n=15). Here, E. coli was isolated in lung tissue of 9 cases. P. multocida and P. aeruginosa, S. aureus and S. uberis, or Nocardia spp. were detected in lung tissue of one case. In lung tissue of three other cases with pneumonia, no pathogen was isolated. Enteritis due to infections with E. coli (n=2) and C. perfringens (n=1) were diagnosed in 3 cases.

### Virology

All animals with haemorrhagic disease were tested for BVDV and BTV. Neither viral antigens nor the presence of the viral genomes could be demonstrated for either of these viral agents (data not shown).

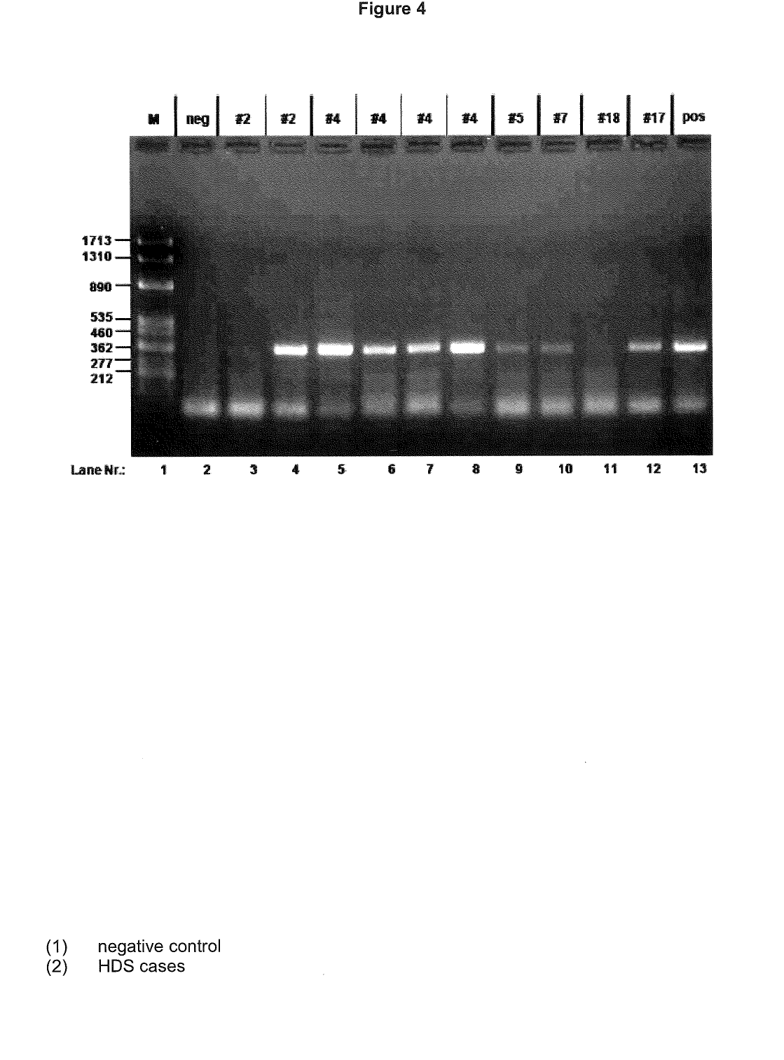

Organ tissues collected from 25 cases (Nos. 1, 2, 4-13, 15-22, 31, 34, 41, 42, 45), were investigated for the presence of circoviral DNA by nested broad-spectrum PCR, using primers with binding sites in the ORF-V1 of the circovirus genome. In samples tested positive, agarose gel electrophoresis revealed bands with the expected length of approximately 350 bp. FIG. 4 shows a negative bone marrow sample of case No. 2 (lane 3), whereas a strong band with the expected size had formed when blood was analyzed (lane 4).

In case No. 4, bone marrow, liver, kidney and blood were positive (lanes 5-8). Weaker bands were detected when samples collected from other calves were investigated (lanes 9, 10 and 12); others remained negative (line 11). In total, 5 out of 25 cases of the study group (Nos. 2, 4, 5, 7 17) and 1 out of 8 controls tested positive in the circovirus PCR. The PCR products of three samples (calves Nos. 2, 4 and 17) were sequenced, and identities of 99% were obtained when compared with nucleotide sequences of PCV2 present in the GenBank database. Out of the 25 samples under investigation, the five samples tested positive in circovirus-specific PCR plus four randomly selected out of the samples tested negative were sent to another laboratory; a routinely used PCV2-specific PCR protocol revealed negative results in all cases (data not shown).

### Whole Genome Sequence Analysis of Strain PCV2-Ha08

Based on the sequence of the PCR products, inverse primers were created which were capable of amplifying the complete circovirus genome present in the sample of case No. 4 tested positive with bone marrow, liver, kidney and blood. The strain was designated as PCV2-Ha08 and completely sequenced. The PCV2-Ha08 genome has a length of 1768 nucleotides. Sequence analysis revealed three ORFs with similarities to the PCV2 Rep and capsid protein and to the product of ORF3. The stem-loop structure, 11 bp in size and containing the conserved nonamer sequence, is evident in the non-coding region 1 (NCR1).

A sequence similarity search of the PCV2-Ha08 genome sequence with sequences of the GenBank database revealed the highest degree of identity (99%) with PCV2 isolate DK558control (EF565365), originating from a pig in Denmark. Comparison of the deduced amino acid sequences of the Rep, Cap and ORF3 product with that of selected porcine and bovine circoviruses revealed identities between 68.5% and 100% (Tab. 3). In all cases, PCV2-Ha08 was closely related to PCV2b-strains and showed the highest percentages of identity with isolate DK558control (EF565365).

A phylogenetic analysis was performed using the whole genome sequences of PCV2-Ha08, the bovine circovirus (AF109397), ten circoviruses sharing highest sequence similarity (determined by BLAST search), and three reference strains defining subtypes PCV2a, PCV2b and PCV2c. (Segales, et al., 2008) As shown in the phylogenetic tree (FIG. 6), PCV2-Ha08 clearly clusters within the PCV2b subtype, however, it forms a separate branch within this group. In contrast, the bovine circovirus (AF109397), which had been previously described to infect cattle in Canada, clusters together with PCV2a.

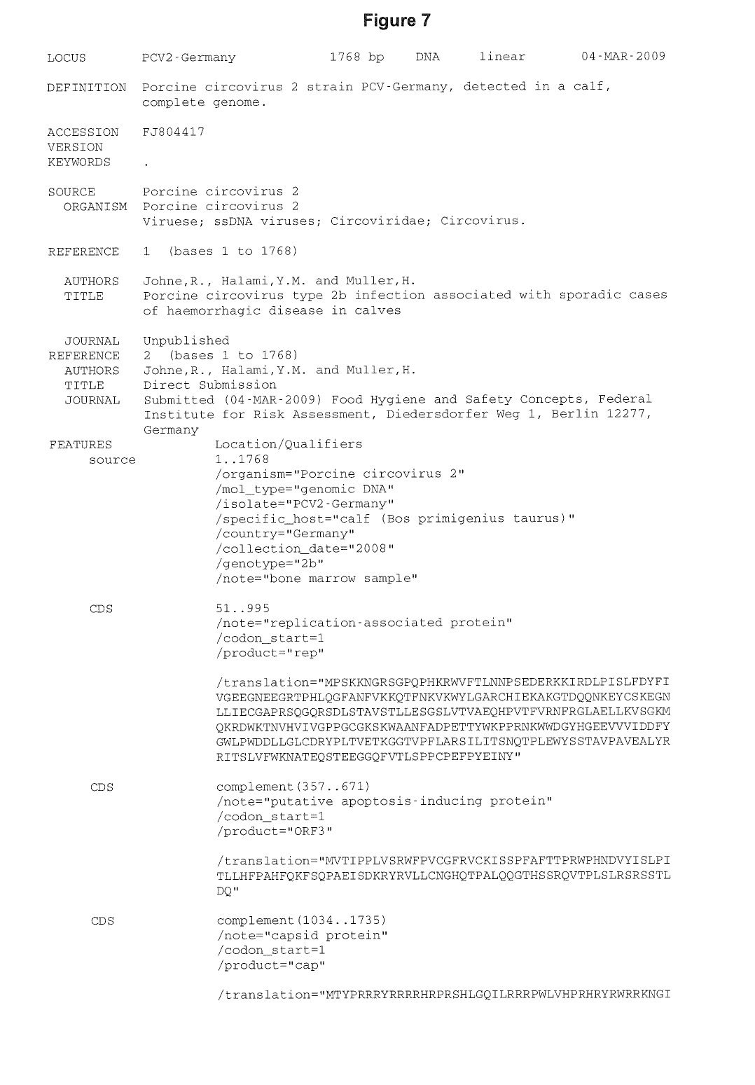

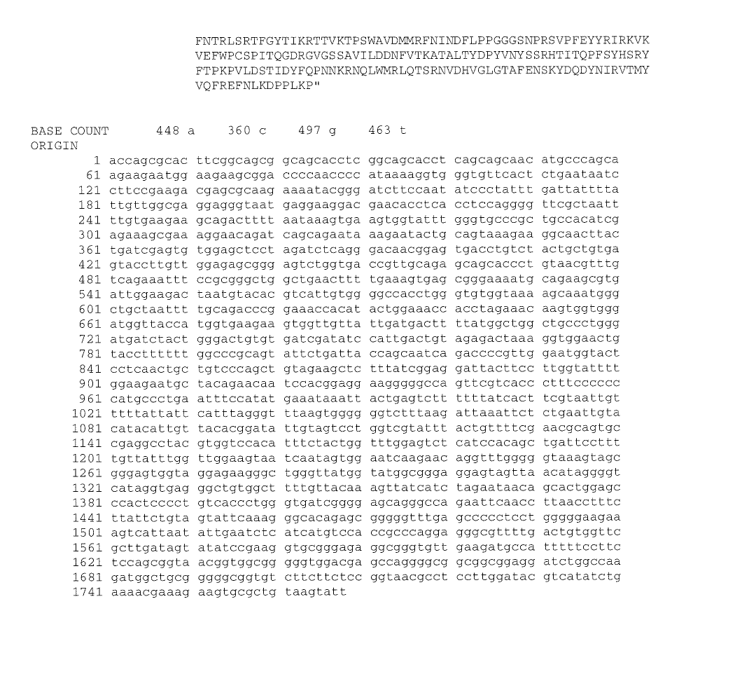

The nucleotide sequence and amino acid sequence of 3 open reading frames of PCV2-Ha08 is shown in FIG. 7 and in the sequence listing (SEQ ID NO; 1, 2, 3 and 4).

### Isolation of PCV2-Ha09 and PCV2-Hal 0

Two further isolates were obtained according to the protocol described supra (see section “Amplification of the whole circovirus genome”).

An isolate obtained from the blood of calf from Bavaria which had died under the symptoms of bovine neonatal pancytopenia (BNP) was designated PCV2-Ha09 and completely sequenced. Analogous to the PCV2-Ha08 strain, the PCV2-Ha09 genome has a length of 1768 nucleotides and contains three ORFs with similarities to the PCV2-Rep and capsid protein and to the product of ORF3.

An isolate from lung and brain of a calf from Saxonia, which had died from BNP as well, was designated as PCV2-Ha10 and completely sequenced. The PCV2-Ha10 genome has a length of 1767 nucleotides and as PCV2-Ha08 and PCV2-Ha09 contains three ORFs with similarities to the PCV2-Rep and capsid protein and to the product of ORF3.

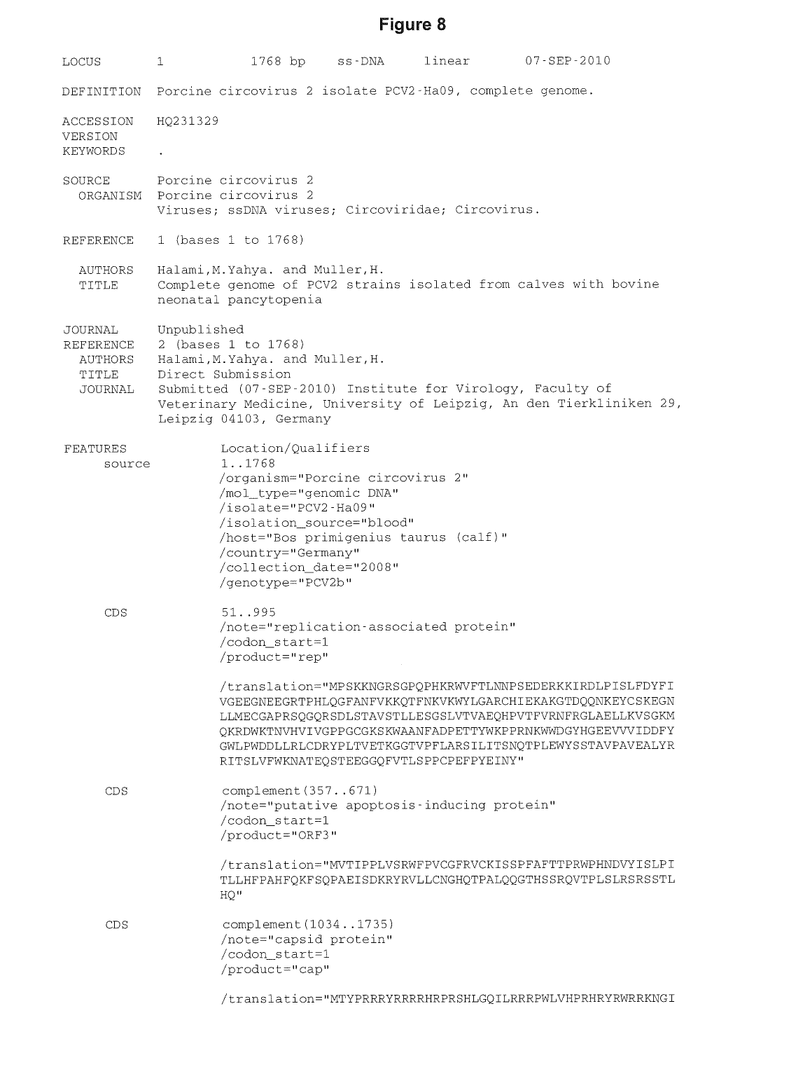

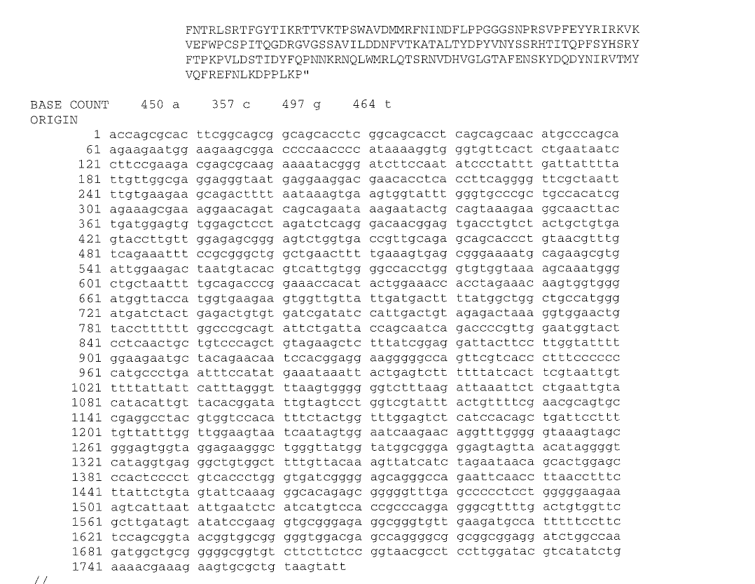

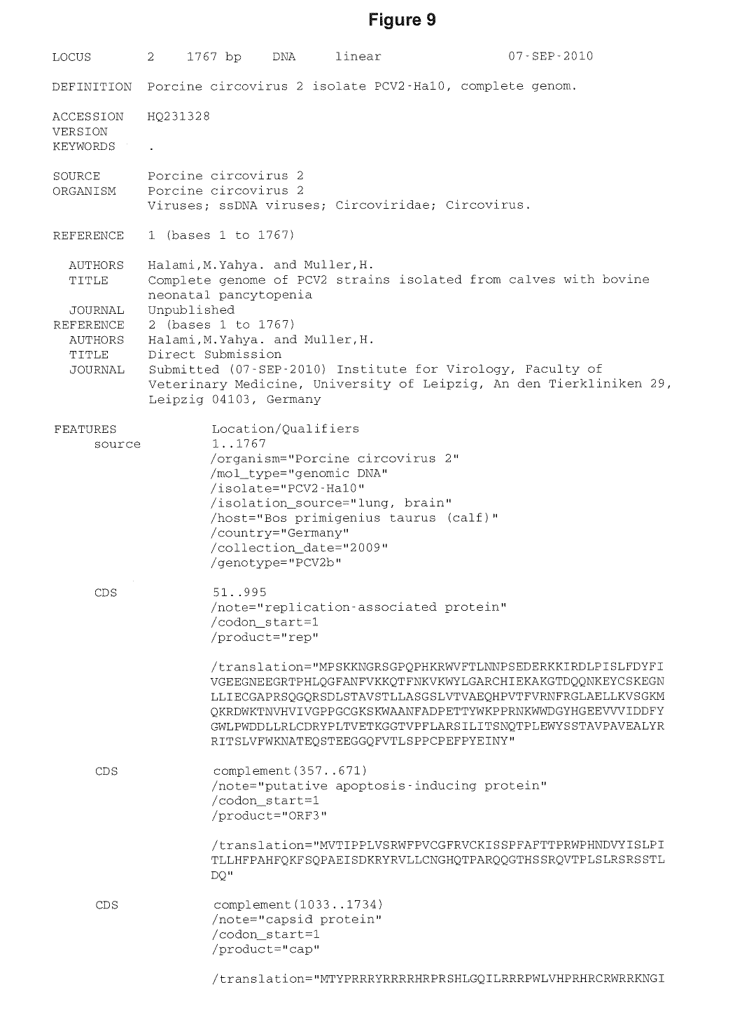

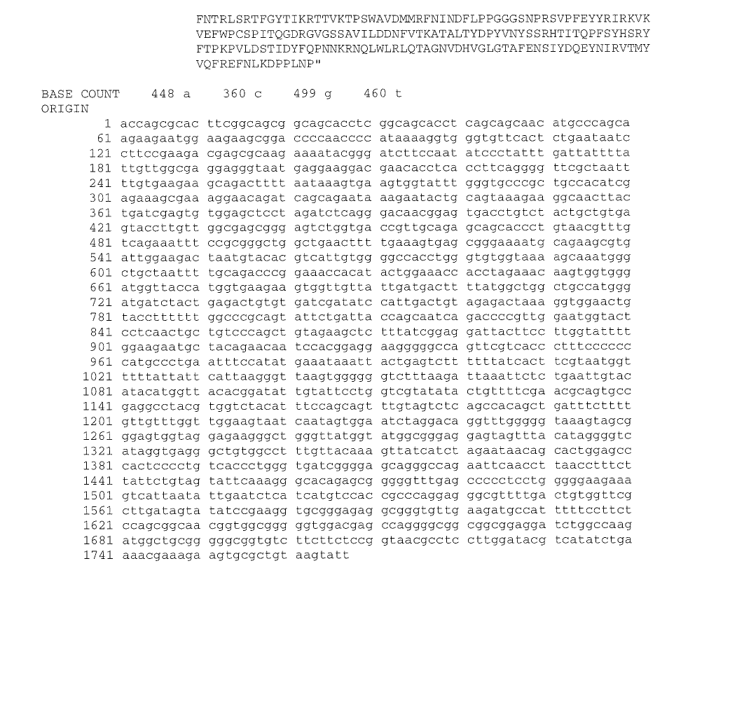

The nucleotide sequences of PCV2-Ha09 and PCV-Ha10 as well as the amino acid sequences of three open reading frames for each of PCV2-Ha09 and PCV-Ha10 are shown in FIGS. 8 and 9 and in the sequence listing (PCV2-Ha09: SEQ ID NOs. 7, 8, 9 and 10; PCV2-Ha10: SEQ ID NOs. 11, 12, 13, and 14).

### Immunohistochemistry

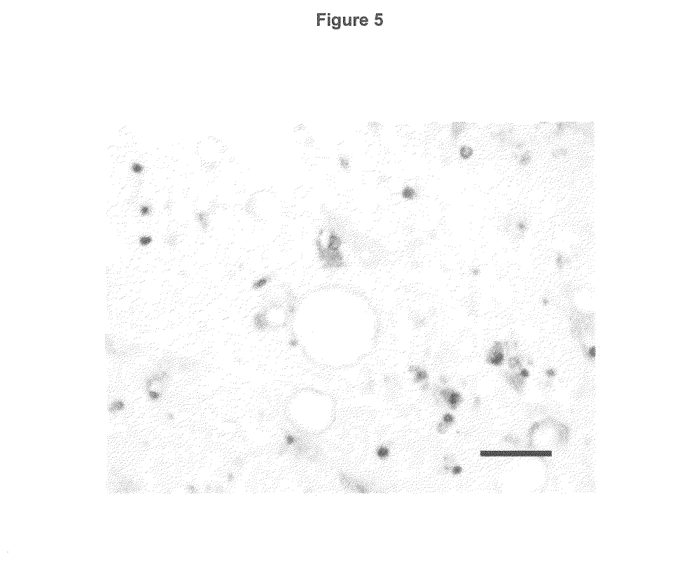

Immunohistochemistry (IHC) was performed on tissue sections of bone marrow, spleen, and lymph node of 2 cases (Nos. 1 and 3) to detect PCV2 antigen. Only single bone marrow cells of case No. 1 showed mild immunoreactivity (FIG. 5). All tissues of case No. 3 and lymphatic tissues of case No. 1 were negative for PCV2 antigen.

### Detection of PCV2-Specific Antibodies in Cattle

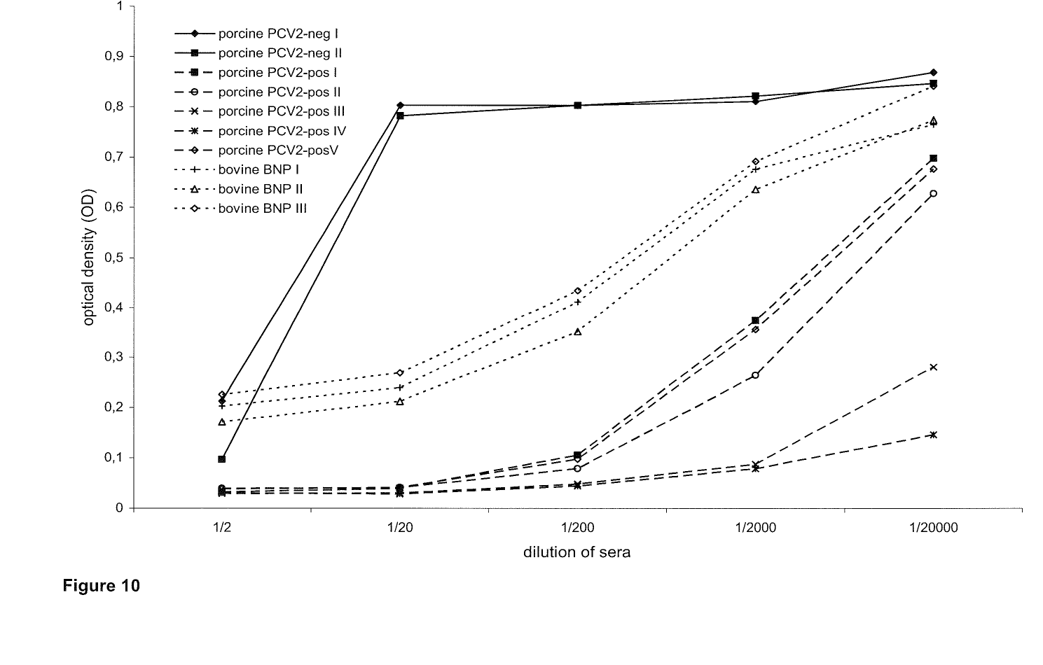

Detection of PCV2 by polymerase chain reaction (PCR) in Bovine Neonatal Pancytopenia (BNP) affected calves raised the question for its role in BNP pathogenesis. If PCV2 is a major player in the pathogenesis of BNP one should be able to detect PCV2-specific antibody responses in affected herds. We tried to detect PCV2-specific antibodies in cattle by a competitive approach: ELISA-plates coated with PCV2-ORF2-antigens were incubated with dilutions (1/2, 1/20, 1/200, 1/2000, 1/20000) of serum in PBS for 90 minutes at 37° C. in a humidified chamber. Test plates were rinsed three times with PBS. An anti-PCV2 monoclonal antibody (mab) conjugated to horse radish peroxidase was diluted 1/500 in PBS and added to the test plate. It incubated another 90 minutes at 37° C. in a humidified chamber. After rinsing the test plate with PBS substrate (TMB/H₂O₂) was added. Colour development was stopped by addition of H₂SO₄ and optical densities (OD) were measured at 450 nm. As references the positive controls of two commercialized testkits were included (Synbiotics, Ingenasa). Wells without serum (PBS) served as negative reference. Two sera from PCV2-negative piglets, five sera from PCV2 positive piglets and three bovine sera from BNP affected herds were included. Results are summarized in FIG. 10. From a dilution 1/20 onwards PCV2-negative and positive porcine sera were clearly discriminated. Three bovine sera from a BNP affected herd inhibited the binding of the PCV2-specific mab in a dose-dependent manner. Although no complete inhibition as for porcine sera was observed, the inhibition exceeded that of the positive references (e.g. Ingenasa PC OD=0.343). These data are highly suggestive for a PCV2-specific immune response in cattle.

### Discussion

Here we describe a haemorrhagic diathesis (HD, also referred to as haemorrhagic disease syndrome (HDS) and bovine neonatal pancytopenia (BNP)) of calves, which could be distinguished from other haemorrhages by following clinical, pathological and histological criteria: The most prominent clinical signs were spontaneous transcutaneous bleedings without obvious injury, haemorrhages in mucosal surfaces and excessive bleeding associated with standard management procedures. Consistently, the haemorrhagic disease became evident in young calves within their first month of life. Severe bone marrow hypoplasia to aplasia was found in all cases. The haematological results indicating aplastic pancytopenia in five of these animals supported this finding. The resulting thrombocytopenia causing the haemorrhagic disease is believed to represent the major pathomechanism of the disease. Furthermore, the haematologic results revealed moderate to severe leucopenia and granulocytopenia. This finding is consistent with the severe bone marrow depletion observed in all animals and depletion of lymphatic tissues in 43% of the animals. The lack of proliferating lymphatic cells is supposed to cause immunosuppression. This may explain the frequent occurrence of lesions such as pneumonia and ulcerative stomatitis as well as the lack of inflammatory cells in some of these lesions.

Following bone marrow destruction, the onset of clinical signs will largely depend on the half-life of blood cells in the circulation, especially of platelets. Anaemia is less significant, unless complicated by bleeding, due to the long life span of bovine erythrocytes of 120 days (Loesch, et al., 2000, Valli, 2007). Platelets life span is merely 9 days and with only 8-9 h half-life of neutrophils in the circulation is even shorter (Paape, et al., 2003, Valli, 2007). Taking these facts into consideration, we hypothesize that the destructive insult may occur in the neonatal calf.

To assess the aetiology of HD, several causes of haemorrhages in cattle due to thrombocytopenia were investigated. Hereditary haemorrhagic diathesis is described in Simmental cattle and is known as Simmental hereditary thrombopathy. It is caused by a marked dysfunction of platelets (Steficek, et al., 1993). Here, Simmental cattle were affected in most cases, but two Holstein Friesian calves showed equivalent lesions. In southern Germany, Simmental cattle are the most common breed and may, therefore, be overrepresented in this study. The distinct clinical picture of the disease in different breeds and the results of pedigree analysis indicate no autosomal dominant or recessive hereditary disease. However, the number of animals in the study was not sufficient to make a definitive statement at present.

Infection with non-cytopathic type 2 BVDV may result in severe bleeding tendency due to thrombocytopenia (Ellis, et al., 1998, Rebhun, et al., 1989). Current thought is that decrease in the maturation pool of bone marrow, decreased numbers of circulating platelets, and altered platelet function contribute to haemorrhages (Ellis, et al., 1998, Walz, et al., 2001, Wood, et al., 2004). However, the bone marrow cellularity does not decrease in BVDV infections. In contrast, severe bone marrow depletion was a constant finding in the cases reported in this study. Furthermore, BVDV was not detected in any of the calves under investigation. On this account, it seems reasonable to exclude a BVDV infection.

Several toxins and mycotoxins are known to cause haemorrhages in cattle. The medical history of the diseased calves and information about animal keeping gave some indication for possible intoxication in individual cases, for example with mycotoxins or drugs. However, there was no consistency in given information which would apply to all affected farms and give reason to suspect a specific toxin. Nevertheless, some random tests for toxins were carried out and remained negative. Particularly, intoxications with S-(1,2-Dichlorovinyl)-L-cysteine (DCVC) or furazolidone, both causing bone marrow aplasia and haemorrhages, fit to the observed lesions. Trichloroethylene-extracted soybean oil meal fed to calves produces fatal aplastic anaemia and renal injury at higher doses. DCVC, a metabolite of trichloroethylene, is the toxic factor in this entity. Experimentally, low doses of DCVC (0.4 mg/kg per day i.v.) administered for 10 days resulted in a marked acellularity of bone marrow and extensive haemorrhages (Lock, et al., 1996). Currently, hexane instead of trichloroethylene is used for extraction of soybean oil. Testing of blood, renal tissue and urine of a total of 4 calves for trichloroethylene, DCVC, and its metabolite N-acetyl-DCVC yielded negative results. The antibiotic furazolidone is used for treatment or prophylaxis of bacterial and protozoan infections in human and animals. Experimentally, daily dosages of 4.0 to 8.0 mg furazolidone per kg bodyweight administered to milk fed calves produce fatal haemorrhagic diathesis due to severe bone marrow depletion (Hoffmann-Fezer, et al., 1974, Hofmann, et al., 1974). According to a national council regulation, the administration of furazolidone to food producing animals is prohibited. Anyhow, 3 affected calves were investigated and proved to be negative with regard to furazolidone.

Ingestion of bracken fern (Pteridium aquilinum) causes symptoms of poisoning in grazing animals. Acute bracken fern poisoning in cattle produces irreversible bone marrow hypoplasia resulting in aplastic pancytopenia. Chronic ingestion leads to enzootic haematuria and is associated with tumours in the lower urinary tract and the alimentary tract (Maxie and Newman, 2007, Valli, 2007). Also, intoxications with mycotoxins of Stachybotrys chartarum are described in ruminants and horses as pancytopenic disease (Harrach, et al., 1983, Valli, 2007). Bracken fern poisoning and stachybotryotoxicosis seem to be unlikely in these cases because symptoms should emerge in animals of all ages and especially in those fed with roughage-containing diet. In this study, forage samples were tested negative for mycotoxins and there was no indication for the uptake of bracken fern by calves or cows.

Idiopathic thrombocytopenic purpura is described as a rare condition in cows (Yeruham, et al., 2003). The cause of this autoimmune disease may be immune-mediated destruction of platelets (Lunn and Butler, 1991). Reported cases of thrombocytopenic purpura were associated with a recent multivalent botulism toxoid vaccination or inactivated vaccines against papilloma virus and clostridia, respectively (Lunn and Butler, 1991, Yeruham, et al., 2003). Calves in this study were not vaccinated. Furthermore, the occurrence of bone marrow destruction is inconsistent with the described immune-mediated thrombocytopenia in cows. Infections with P. multocida types B or E are known to cause haemorrhagic septicaemia in calves (Rimler, 1978). Endotoxins play a major role in the pathogenesis of this infection (Horadagoda, et al., 2001). In this study, P. multocida was present in only three calves. A typing was not carried out. In our study, microbiological investigation of organ samples revealed a wide spectrum of potentially pathogenic bacteria in diseased calves. However, constant evidence of a specific pathogenic bacteria associated with the haemorrhagic disease could not be found. In 29% (n=16) of all cases, no pathogenic bacteria were detected. In 17 calves isolated bacteria were associated with additional inflammatory lesions such as pneumonia or enteritis. It may be assumed that depletion of lymphatic tissue and bone marrow in these calves resulted in severe leucopenia and granulocytopenia associated with immunosuppression and secondary infection.

Using PCR, a circovirus was detected in some of the clinically diseased calves. Circovirus infection in cattle has not been convincingly described so far. Serological investigations on circovirus-specific antibodies led to contradictory results (Allan, et al., 2000, Ellis, et al., 2001, Tischer, et al., 1995). Only one publication has shown that a circovirus closely related to PCV2 could be detected in lung tissues and fetuses of cattle (Nayar, et al., 1999). Interpretation of PCR results is sometimes difficult, especially with respect to DNA contamination. In our study, however, we implemented a strict regime to exclude laboratory DNA contamination. The successful amplification of the whole PCV2 genome from one sample argues against a contamination with short PCR products. The negative result of the routinely PCV2-specific PCR protocol may be explained by a lower sensitivity of this protocol as compared to the nested protocol of the broad-spectrum PCR.

The analysis of the whole genome sequence of the circovirus PCV2-Ha08 revealed a close relationship to PCV2b. The only circovirus sequence originating from bovine tissue (Nayar, et al., 1999) and available at the GenBank database is also closely related to PCV2. However, the detailed analysis shows that both strains cluster into different subtypes thus excluding the existence of a distinct PCV2 strain which is able to infect cattle. In the meantime, two more strains, PCV2-Ha09 and PCV2-Ha10 have been isolated. Circoviruses are generally thought to have narrow host ranges and detailed phylogenetic analyses revealed a strict coevolution of circoviruses with their hosts (Johne, et al., 2006). For PCV2, however, a slightly different evolutionary and epidemiological pattern has been described, which is consistent with a prolonged period of limited transmission in the past followed by a recent worldwide spread of this virus (Hughes and Piontkivska, 2008). It may be speculated that PCV2 has accessed specific properties allowing rapid spread and—in rare cases—transmission across the species barrier.

In addition, PCV2 was detected in one of the calves from the control group. Control animals had been sent for pathological examination for reasons other than haemorrhagic disease. PCV2 is associated with different syndromes and diseases in pigs. According to this, it may be speculated that circoviruses contribute to several diseases in calves. It is also conceivable that immunosuppression in calves with HD enhances susceptibility to other infections. In this case, detection of PCV2 in calves may reflect an opportunistic infection. Finally it is well-known that circovirus infections may remain clinically inapparent for various periods of time, or even over the total life-span of an infected individual.

An infection with a circovirus would be consistent with many of the observed clinical signs as most of the circoviruses cause lymphocyte depletion and the related CIAV also causes aplastic anaemia and haemorrhages in infected chicken. In our study, however, PCV2 was not detected in all clinical cases using the available diagnostic methods. Also, detection by PCR does not necessarily mean infection with a replicating virus. Detection of PCV2 antigen by immunohistochemistry in some individual bone marrow cells is, however, indicative of viral genome expression and replication.

### TABLES

TABLE 1
Characteristics of animals of case and control groups including
detection of circoviral genomic sequences by PCR and final diagnosis

| | | Age | | | Weight | | |
|---|---|---|---|---|---|---|---|
| Group | No. | (d) | Sex | Breed | (kg) | Circovirus | Diagnosis |
| case | 1 | 17 | M | SC | 56 | neg | HDS |
| case | 2 | 18 | M | SC | 55 | pos (b) | HDS |
| case | 3 | 15 | F | SC | 52 | n.d. | HDS |
| case | 4 | 0 | M | SC | 58 | pos (b, bm, l, k) | HDS |
| case | 5 | 20 | F | SC | 42 | pos (b) | HDS |
| case | 6 | 15 | F | SC | 45 | neg | HDS |
| case | 7 | 0 | F | NN | 55 | pos (bm) | HDS |
| case | 8 | 28 | M | HF | 48 | neg | HDS |
| case | 9 | 20 | M | HF | 44 | neg | HDS |
| case | 10 | 19 | M | SC | 50 | neg | HDS |
| case | 11 | 17 | F | SC | 47 | neg | HDS |
| case | 12 | 16 | F | SC | 55 | neg | HDS |
| case | 13 | 0 | NN | NN | 60 | neg | HDS |
| case | 14 | 0 | NN | NN | 52 | n.d. | HDS |
| case | 15 | 25 | F | SC | 41 | neg | HDS |
| case | 16 | 16 | M | SC | 60 | neg | HDS |
| case | 17 | 17 | M | NN | 60 | pos (bm) | HDS |
| case | 18 | 15 | F | SC | 50 | neg | HDS |
| case | 19 | 18 | F | SC | 52 | neg | HDS |
| case | 20 | 11 | F | SC | 52 | neg | HDS |
| case | 21 | 17 | F | SC | 58 | neg | HDS |
| case | 22 | 7 | M | SC | 46 | neg | HDS |
| case | 23 | 18 | F | SC | 52 | n.d. | HDS |
| case | 24 | 10 | M | SC | 58 | n.d. | HDS |
| case | 25 | 20 | F | SC | 51 | n.d. | HDS |
| case | 26 | 20 | M | SC | 61 | n.d. | HDS |
| case | 27 | 19 | F | SC | 42 | n.d. | HDS |
| case | 28 | 16 | M | SC | 51 | n.d. | HDS |
| case | 29 | 11 | M | SC | 50 | n.d. | HDS |
| case | 30 | 14 | M | SC | 62 | n.d. | HDS |
| case | 31 | 23 | M | SC | 63 | n.d. | HDS |
| case | 32 | 0 | M | SC | 46 | n.d. | HDS |
| case | 33 | 0 | M | SC | 72 | n.d. | HDS |
| case | 34 | 32 | M | SC | 57 | n.d. | HDS |
| case | 35 | 16 | F | SC | 58 | n.d. | HDS |
| case | 36 | 19 | F | SC | 66 | n.d. | HDS |
| case | 37 | 10 | F | SC | 53 | n.d. | HDS |
| case | 38 | 14 | M | SC | 51.5 | n.d. | HDS |
| case | 39 | 16 | M | SC | 40 | n.d. | HDS |
| case | 40 | 18 | M | SC | 56 | n.d. | HDS |
| case | 41 | 14 | F | SC | 48 | n.d. | HDS |
| case | 42 | 25 | M | SC | 60 | n.d. | HDS |
| case | 43 | 20 | M | SC | 45 | n.d. | HDS |
| case | 44 | 16 | M | SC | 46 | n.d. | HDS |
| case | 45 | 14 | F | SC | 34 | n.d. | HDS |
| case | 46 | 21 | F | SC | 70 | n.d. | HDS |
| case | 47 | 13 | M | SC | 58 | n.d. | HDS |
| case | 48 | 13 | M | SC | 57 | n.d. | HDS |
| case | 49 | 15 | F | SC | 60 | n.d. | HDS |
| case | 50 | 7 | F | SC | 49 | n.d. | HDS |
| case | 51 | 19 | M | SC | 57 | n.d. | HDS |
| case | 52 | 21 | M | SC | 56.5 | n.d. | HDS |
| case | 53 | 19 | w | SC | 43 | n.d. | HDS |
| case | 54 | 11 | w | mixed | 43 | n.d. | HDS |
| case | 55 | 15 | w | SC | 50 | n.d. | HDS |
| case | 56 | 8 | w | SC | 50 | n.d. | HDS |
| control | 1 | 0 | F | SC | 42 | neg | abortion |
| control | 2 | n.n. | F | SC | 46 | neg | gastroenteritis |
| control | 3 | 18 | F | SC | 42 | neg | polyarthritis |
| control | 4 | n.n. | F | SC | 38 | neg | enteritis |
| control | 5 | 9 | F | SC | 46 | neg | enteritis, dehydration, pneumonia |
| control | 6 | n.n. | F | SC | 30 | neg | enteritis, dehydration |
| control | 7 | 8 | M | SC | 49 | pos | abomasoenteritis, dehydration |
| control | 8 | 18 | F | SC | 34 | neg | enteritis, dehydration, bronchitis |

HDS: Haemorrhagic Disease Syndrome;
M: male;
F: female;
SC: Simmental cattle;
neg: negative;
pos: positive;
n.n.: not named;
b: blood,
bm: bone marrow;
l: liver;
k: kidney

TABLE 2
Haematologic results of five calves with haemorrhagic disease and
bone marrow aplasia

| | Reference | | | No. | No. | No. | No. |
|---|---|---|---|---|---|---|---|
| | Range | Unit | No. 2 | 53 | 54 | 55 | 56 |
| Haemo- | 5.6-8.7 | mmol/l | 4.9 | 1.2 | 5.3 | 2.65 | 4.5 |
| globin | | | | | | | |
| Haematocrit | 0.22-0.44 | I/l | 0.23 | 0.06 | 0.27 | 0.13 | 0.22 |
| Erythrocytes | 5.0-9.7 | T/l | 6.1 | 1.32 | 6.3 | 2.94 | 5.6 |
| White Blood | 4.0-12.0 | G/l | 0.85 | 1.47 | 0.285 | 0.9 | 1.0 |
| Cell Count | | | | | | | |
| MCV | 36-50 | fl | 37.3 | 42.1 | 43.9 | 43.2 | 39.5 |
| MCH | 13-17 | pg | 12.9 | 14.6 | 13.7 | 14.5 | 13.0 |
| MCHC | 32-39 | mmol/l | 21.5 | 21.5 | 19.4 | 20.9 | 20.4 |
| Neutrophil | 25-45 | % | 1 | 4 | 28 | 3 | 4 |
| granulocytes | | | | | | | |
| Eosinophil | 0-14 | % | 1 | 0 | 0 | 0 | 0 |
| granulocytes | | | | | | | |
| Basophil | 0-1 | % | 1 | 0 | 0 | 2 | 0 |
| granulocytes | | | | | | | |
| Monocytes | 1-8 | % | 1 | 2 | 4 | 11 | 7 |
| Lympho- | 45-65 | % | 96 | 94 | 68 | 84 | 89 |
| cytes | | | | | | | |
| Platelet | 300-800 | G/l | 33.7 | 12.5 | 82 | 60 | 55 |
| count | | | | | | | |

TABLE 3
Comparison of sequence similarities between PCV2-Ha08 and
other circoviruses.

| | PCV2- | PCV2- | PCV2- | PCV2- | PCV2- | PCV2-Ha08/ |
|---|---|---|---|---|---|---|
| | Ha08/ | Ha08/ | Ha08/ | Ha08/ | Ha08/ | PCV2 |
| | PCV1 | PCV2a | PCV2b | PCV2c | BCV | DK558control |
| Ge- | 78% | 96.3% | 98.9% | 94.7% | 96.1% | 99% |
| nome¹ | | | | | | |
| Rep² | 86.5% | 99.4% | 99.4% | 99.4% | 99.4% | 99.7% |
| Cap² | 68.5% | 94% | 97% | 87.2% | 91.9% | 97.4% |
| ORF-3² | — | 96.2% | 99% | 97.1% | 96.2% | 100% |
| NCR1¹ | 84.8% | 98.8% | 100% | 98.8% | 98.8% | 100% |
| NCR2¹ | 78.9% | 97.4% | 97.3% | 97.1% | 97.4% | 97.3% |

The accession numbers of the used sequences are the same as in FIG. 6.
¹nucleotide sequence
²amino acid sequence

### REFERENCES

Allan G, McNeilly F, Krakowa S, Ellis J (2003): PCV-2 infection in swine; more than just postweaning multisystemic wasting syndrome. Vet J 166: 222.
Allan G M, McNeilly F, McNair I, Curran M D, Walker I, Ellis J, Konoby C, Kennedy S, Meehan B (2000): Absence of evidence for porcine circovirus type 2 in cattle and humans, and lack of seroconversion or lesions in experimentally infected sheep. Arch Virol 145: 853.
Chae C (2005): A review of porcine circovirus 2-associated syndromes and diseases. Vet J 169: 326.
Cox, J. C. and Coulter, A. R. (1992): “Advances in Adjuvant Technology and Application”, in Animal Parasite Control Utilizing Biotechnology, Chapter 4, Ed. Yong, W. K., CRC Press.
Dalsgaard, K. (1974): Saponin adjuvants. 3. Isolation of a substance from Quillaja saponaria Molina with adjuvant activity in food-and-mouth disease vaccines. Arch Gesamte Virusforsch 44: 243.
Ellis J, Krakowka S, Lairmore M, Haines D, Bratanich A, Clark E, Allan G, Konoby C, Hassard L, Meehan B, Martin K, Harding J, Kennedy S, McNeilly F (1999): Reproduction of lesions of postweaning multisystemic wasting syndrome in gnotobiotic piglets. Vet Diagn Invest 11: 3.
Ellis J A, Konoby C, West K H, Allan G M, Krakowka S, McNeilly F, Meehan B, Walker I (2001): Lack of antibodies to porcine circovirus type 2 virus in beef and dairy cattle and horses in western Canada. Can Vet J 42: 461.
Ellis J A, West K H, Cortese V S, Myers S L, Carman S, Martin K M, Haines D M (1998): Lesions and distribution of viral antigen following an experimental infection of young seronegative calves with virulent bovine virus diarrhea virus-type II. Can J Vet Res 62: 161.
Goryo M, Suwa T, Umemura T, Itakura C, Yamashiro S (1989): Histopathology of chicks inoculated with chicken anaemia agent (MSB1-TK5803 strain). Avian Pathol 18: 73.
Gottschalk C, Bauer J, Meyer K (2008): Detection of satratoxin g and h in indoor air from a water-damaged building. Mycopathologia 166: 103.
Halami M Y, Nieper H, Muller H, Johne R (2008): Detection of a novel circovirus in mute swans (Cygnus olor) by using nested broad-spectrum PCR. Virus Res 132: 208.
Harrach B, Bata A, Bajmocy E, Benko M (1983): Isolation of satratoxins from the bedding straw of a sheep flock with fatal stachybotryotoxicosis. Appl Environ Microbiol 45: 1419.
Hoffmann-Fezer G, Hoffmann R, Hofmann W (1974): [Chronic furazolidone poisoning in calves. II. studies of the bone marrow]. Dtsch Tierarztl Wochenschr 81: 59.
Hofmann W, Hoffmann R, Hoffmann-Fezer G (1974): [Chronic furazolidone poisoning in calves. I. Clinical, hematological and morphological studies]. Dtsch Tierarztl Wochenschr 81: 53. Horadagoda N U, Hodgson J C, Moon G M, Wijewardana T G, Eckersall P D (2001): Role of endotoxin in the pathogenesis of haemorrhagic septicaemia in the buffalo. Microb Pathog 30: 171.
Hughes A L, Piontkivska H (2008): Nucleotide sequence polymorphism in circoviruses. Infect Genet Evol 8: 130.
Johne R, Fernandez-de-Luco D, Hofle U, Muller H (2006): Genome of a novel circovirus of starlings, amplified by multiply primed rolling-circle amplification. J Gen Virol 87: 1189.
Kennedy S, Moffett D, McNeilly F, Meehan B, Ellis J, Krakowka S, Allan G M (2000): Reproduction of lesions of postweaning multisystemic wasting syndrome by infection of conventional pigs with porcine circovirus type 2 alone or in combination with porcine parvovirus. J Comp Pathol 122: 9.
Kuscu B, Gurel A (2008): Lesions in the thymus and bone marrow in chicks with experimentally induced chicken infectious anemia disease. J Vet Sci 9:15.
Liu J, Chen I, Kwang J (2005): Characterization of a previously unidentified viral protein in porcine circovirus type 2-infected cells and its role in virus-induced apoptosis. J Virol 79: 8262.
Lock E A, Sani Y, Moore R B, Finkelstein M B, Anders M W, Seawright A A (1996): Bone marrow and renal injury associated with haloalkene cysteine conjugates in calves. Arch Toxicol 70: 607.
Loesch U, Cihak J, Erhard M H, Kaspers B (2000): Blut and Abwehr. In: von Engelhardt W, Breves G (eds) Physiologie der Haustiere. Enke im Hippokrates Verlag GmbH, Stuttgart, Germany p 196.
Lunn D P, Butler D G (1991): Idiopathic thrombocytopenic purpura in a Holstein bull. Can Vet J 32: 559.
Mankertz A (2008): Circoviruses. In: Mahy B W J, Van Regenmortel M H V (eds) Encyclopedia of Virology, vol 1. Elsevier Books, Amsterdam p
Maxie M G, Newman S J (2007): Urinary system. In: Maxie M G (ed) Jubb, Kennedy, and Palmer's Pathology of domestic animals, vol 2. Elsevier, Amsterdam p 425.
Nayar G P, Hamel A L, Lin L, Sachvie C, Grudeski E, Spearman G (1999): Evidence for circovirus in cattle with respiratory disease and from aborted bovine fetuses. Can Vet J 40: 277.
Paape M J, Bannerman D D, Zhao X, Lee J W (2003): The bovine neutrophil: Structure and function in blood and milk. Vet Res 34: 597.
Rebhun W C, French T W, Perdrizet J A, Dubovi E J, Dill S G, Karcher L F (1989): Thrombocytopenia associated with acute bovine virus diarrhea infection in cattle. J Vet Intern Med 3: 42.
Reubel G H, Gareis M, Amselgruber W M (1987): Cytotoxicity evaluation of mycotoxins by an MTT-bioassay. Mycotoxin Res 3: 85.
Rhoades K R, Heddleston K L, Rebers P A (1967): Experimental hemorrhagic septicemia: gross and microscopic lesions resulting from acute infections and from endotoxin administration. Can J Comp Med Vet Sci 31: 226.
Rimier R B (1978): Coagglutination test for identification of Pasteurella multocida associated with hemorrhagic septicemia. J Clin Microbiol 8: 214.
Segales J, Allan G M, Domingo M (2005): Porcine circovirus diseases. Anim Health Res Rev 6: 119.
Segales J, Mateu E (2006): Immunosuppression as a feature of postweaning multisystemic wasting syndrome. Vet J 171: 396.
Segales J, Olvera A, Grau-Roma L, Charreyre C, Nauwynck H, Larsen L, Dupont K, McCullough K, Ellis J, Krakowka S, Mankertz A, Fredholm M, Fossum C, Timmusk S, Stockhofe-Zurwieden N, Beattie V, Armstrong D, Grassland B, Baekbo P, Allan G (2008): PCV-2 genotype definition and nomenclature. Vet Rec 162: 867.
Steficek B A, Thomas J S, Baker J C, Bell T G (1993): Hemorrhagic diathesis associated with a hereditary platelet disorder in Simmental cattle. J Vet Diagn Invest 5: 202.
Steinfeldt T, Finsterbusch T, Mankertz A (2001): Rep and Rep′ protein of porcine circovirus type 1 bind to the origin of replication in vitro. Virology 291: 152.
Thompson J D, Higgins D G, Gibson T J (1994): CLUSTAL W: improving the sensitivity of progressive multiple sequence alignment through sequence weighting, positionspecific gap penalties and weight matrix choice. Nucleic Acids Res 22: 4673.
Timmusk S, Wallgren P, Brunborg I M, Wikstrom F H, Allan G, Meehan B, McMenamy M, McNeilly F, Fuxler L, Belak K, Podersoo D, Saar T, Berg M, Fossum C (2008): Phylogenetic analysis of porcine circovirus type 2 (PCV2) pre- and postepizootic postweaning multisystemic wasting syndrome (PMWS). Virus Genes 36: 509.
Tischer I, Bode L, Apodaca J, Timm H, Peters D, Rasch R, Pociuli S, Gerike E (1995): Presence of antibodies reacting with porcine circovirus in sera of humans, mice, and cattle. Arch Virol 140: 1427.
Todd D (2000): Circoviruses: immunosuppressive threats to avian species: a review. Avian Pathol 29: 373.
Todd D, Bendinelli M, Biagini P, Hino S, Mankertz A, Mishiro S, Niel C, Okamoto H, Raidal S, Ritchie B W, Teo G C (2005): Circoviridae. In: Fauquet C M, Mayo M A, Maniloff J, Desselberger U, Ball L A (eds) Virus Taxonomy, VIIIth Report of the International Committee for the Taxonomy of Viruses. Elsevier/Academic Press, Amsterdam p 327.
Toussaint J F, Sailleau C, Breard E, Zientara S, De Clercq K (2007): Bluetongue virus detection by two real-time RT-qPCRs targeting two different genomic segments. J Virol Methods 140: 115.
Valli V E O (2007): Hematopoietic system. In: Maxie M G (ed) Jubb, Kennedy, and Palmer's Pathology of domestic animals, vol 3. Elsevier, Amsterdam p 107.
Walz P H, Bell T G, Grooms D L, Kaiser L, Maes R K, Baker J C (2001): Platelet aggregation responses and virus isolation from platelets in calves experimentally infected with type I or type II bovine viral diarrhea virus. Can J Vet Res 65: 241.
Wood R D, Goens S D, Carman P S, Deregt D, Jefferson B, Jacobs R M (2004): Effect on hematopoietic tissue of experimental infection of calves with noncytopathic type 2 bovine viral diarrhea virus. Can J Vet Res 68: 42.
Yeruham I, Avidar Y, Harrus S, Fishman L, Aroch I (2003): Immune-mediated thrombocytopenia and putative haemolytic anaemia associated with a polyvalent botulism vaccination in a cow. Vet Rec 153: 502.
Yuasa N, Taniguchi T, Yoshida I (1979): Isolation and some characteristics of an agent inducing anemia in chicks. Avian Dis 23: 366.

1. A circovirus (CV) nucleic acid molecule comprising
(a) the sequence as shown in SEQ ID NO: 1 or a fragment thereof and/or
(b) the complement of the nucleotide sequence according to (a).

2. The nucleic acid molecule according to claim 1 comprising
(a) the sequence as shown in SEQ ID NO: 7 or a fragment thereof and/or
(b) the complement of the nucleotide sequence according to (a).

3. The nucleic acid molecule according to claim 1 comprising
(a) the sequence as shown in SEQ ID NO: 11 or a fragment thereof and/or
(b) the complement of the nucleotide sequence according to (a).

4. The nucleic acid molecule according to claim 1, wherein the fragment comprises at least 15, at least 20, at least 25, at least 30 or at least 50 contiguous nucleotides as shown in SEQ ID NOs:1, 7 or 11 or the complement thereof.

5. The nucleic acid molecule according to claim 1
(a) having an identity of at least 90%, at least 92%, at least 94%, at least 96%, at least 98% or at least 99% of the nucleotide sequence as shown in SEQ ID NOs: 1, 7, or 11
(b) hybridizing under stringent conditions to the nucleotide sequence as shown in SEQ ID NOs: 1, 7 or 11 or
(c) the complement of (a) or (b).

6. A CV nucleic acid molecule encoding a CV polypeptide or a fragment thereof, wherein the nucleic acid molecule comprises
(a) a region of the nucleotide sequence as shown in SEQ ID NO: 1 from
(i) nucleotide 51-995 (Rep)
(ii) nucleotide 1034-1735 (Cap)
(iii) nucleotide 357-671 (ORF3)
and/or the complement of (i), (ii) and/or (iii)
(b) a nucleotide sequence corresponding to the sequence of (a) within the scope of degeneracy of the genetic code, or
(c) a fragment of the nucleotide sequence according to (a) or (b).

7. A CV nucleic acid according to claim 6, wherein the nucleic acid molecule comprises
(a) region of the nucleotide sequence as shown in SEQ ID NO: 7 from
(i) nucleotide 51-995 (Rep)
(ii) nucleotide 1034-1735 (Cap)
(iii) nucleotide 357-671 (ORF3)
and/or the complement of (i), (ii) and/or (iii)
(b) a region of the nucleotide sequence as shown in SEQ ID NO: 11 from
(i) nucleotide 51-995 (Rep)
(ii) nucleotide 1033-1734 (Cap)
(iii) nucleotide 357-671 (ORF3)
and/or the complement of (i), (ii) and/or (iii)
(c) a nucleotide sequence corresponding to the sequence of (a) or (b) within the scope of degeneracy of the genetic code, or
(d) a fragment of the nucleotide sequence according to (a), (b) or (c).

8. The nucleic acid molecule of claim 6, wherein the polypeptide or fragment thereof comprises at least 6, at least 8, at least 10, at least 20, or at least 30 contiguous amino acids of any of the amino acid sequences as shown in SEQ ID NO: 2-4, 8-10, or 12-14.

9. A CV nucleic acid according to claim 6, which encodes a polypeptide having an identity of at least 90%, at least 92%, at least 94%, at least 96%, at least 98% or at least 99% of any of the amino acid sequences as shown in SEQ ID NO: 2-4, 8-10, or 12-14.

10. The nucleic acid molecule of claim 1 in operative linkage with a heterologous expression control sequence.

11. A non-human host cell transformed of transfected with a nucleic acid molecule of claim 1.

12. The nucleic acid molecule of claim 1 for use as a diagnostic agent.

13. The nucleic acid molecule of claim 12, which carries a reporter group.

14. The nucleic acid molecule of claim 12 for the diagnosis of Haemorrhagic Diathesis (HD).

15. The nucleic acid molecule of claim 14 for the diagnosis of HD in cattle.

16. The nucleic acid molecule of claim 1 for use as a therapeutic agent.

17. The nucleic acid molecule of claim 16 for the prevention and/or treatment of HD.

18. The nucleic acid molecule of claim 16 for the prevention and/or treatment of HD in cattle.

19. The nucleic acid molecule of claim 16 as a nucleic acid-based vaccine or for the production of a polypeptide-based vaccine.

20. A polypeptide encoded by a nucleic acid molecule according to claim 1.

21. A circovirus (CV) polypeptide comprising
(a) an amino acid sequence selected from
(i) amino acid SEQ ID NO: 2 (Rep)
(ii) amino acid SEQ ID NO: 3 (Cap)
(iii) amino acid SEQ ID NO: 4 (ORF3), or
(b) a fragment thereof.

22. A circovirus (CV) polypeptide according to claim 21, comprising
(a) an amino acid sequence selected from
(i) amino acid SEQ ID NO: 8 and 12 (Rep)
(ii) amino acid SEQ ID NO: 9 and 13 (Cap)
(iii) amino acid SEQ ID NO: 10 and 14 (ORF3), or
(b) a fragment thereof.

23. The polypeptide of claim 20 comprising at least 6, at least 8, at least 10, at least 20, or at least 30 contiguous amino acids of any of the amino acid sequences as shown in SEQ ID NO: 2-4, 8-10, or 12-14.

24. The polypeptide of claim 20 having an identity of at least 90%, at least 92%, at least 94%, at least 96%, at least 98% or at least 99% of any of the amino acid sequences as shown in SEQ ID NO: 2-4, 8-10, or 12-14.

25. The polypeptide of claim 20 for use as a diagnostic agent.

26. The polypeptide of claim 25, which carries a reporter group.

27. The polypeptide of claim 25 for the diagnosis of HD, particularly in cattle.

28. The polypeptide of claim 20 for use as immunogen.

29. The polypeptide of claim 28 for the generation of anti-CV-antibodies.

30. The polypeptide of claim 20 for the prevention and or treatment of HD, particularly in cattle.

31. The polypeptide of claim 30 for use as a vaccine.

32. An antibody or an antigen-binding fragment thereof directed against a polypeptide of claim 20.

33. The antibody of claim 32, which is specific for CV strain PCV2-Ha08.

34. The antibody of claim 32 for use as a diagnostic agent.

35. The antibody of claim 32 for the prevention and/or treatment of HD, particularly in cattle.

36. A circovirus comprising a nucleic acid molecule of claim 1.

37. The virus of claim 36 which is an inactivated circovirus.

38. The virus of claim 37 which is an attenuated circovirus.

39. A composition comprising
a) a nucleic acid molecule comprising the sequence as shown in SEQ ID No: 1 or a fragment thereof and/or the complement of the nucleotide sequence as shown in SEQ ID No: 1,
b) a polypeptide encoded by a nucleic acid molecule of (a),
c) an antibody or an antigen-binding fragment thereof directed against a polypeptide of (b) or
d) a circovirus comprising a nucleic acid molecule of (a), together with an acceptable carrier, diluent and/or adjuvant.

40. The composition of claim 39 which is a vaccine.

41. The composition of claim 40, which is a polypeptide-based vaccine.

42. The composition of claim 40, which is a virus-based vaccine.

43. The composition of claim 39 which is an immunogenic composition.

44. The composition of claim 43 which is a polypeptide-based immunogenic composition.

45. The composition of claim 43 which is a virus-based immunogenic composition.

46. The composition of claim 39 for diagnostic use.

47. The composition of claim 39 for therapeutic use.

48. A method of diagnosing HD, particularly in cattle, comprising:
contacting a sample from a subject to be diagnosed with a diagnostic composition of claim 46 and determining the presence and/or amount of CV or anti-CV antibodies in said sample.

49. A method of preventing or treating HD, particularly in cattle, comprising:
administering to a subject in need thereof the therapeutic composition of claim 47 in an effective amount.
